(19) United States
(12) Patent Application Publication
(10) Pub. No.: US 2005/0221310 A1
(43) Pub. Date: Oct. 6, 2005

(54) Methods for enhancing gene expression analysis

(75) Inventors: Uwe Scherf, Potomac, MD (US); Glenn D. Hoke, Mt. Airy, MD (US); Daniel J. Wilson, Mississauga (CA); Debra A. Barnes, Frederick, MD (US)

Correspondence Address: COOLEY GODWARD LLP;ATTN: PATENT GROUP, 11951 FREEDOM DRIVE, SUITE 1700, ONE FREEDOM SQUARE- RESTON TOWN CENTER, RESTON, VA, 20190-5061, US

(73) Assignee: GENE OGIC, INC., GAITHERSBURG, MD (US)

(21) Appl. No.: 10/504,072

(22) PCT Filed: Jun. 4, 2004
(86) PCT No.: PCT/US04/17621

§ 371 Date: Apr. 11, 2005

Related U.S. Application Data
(60) Provisional application No. 60/476,233, filed on Jun. 6, 2003.
(60) Provisional application No. 60/491,528, filed on Aug. 1, 2003.

(51) Int. Cl. C12Q 1/68; C12P 19/34

(52) U.S. Cl. 435/6; 435/91.2

(57) ABSTRACT

This application concerns improved methods of analyzing gene expression data where mRNA transcripts or representatives thereof that skew the gene expression profile of a cell or tissue sample are identified and removed from the population of mRNA transcripts prior to, during or subsequent to a reverse transcription reaction.

### RELATED APPLICATIONS

This application relates to U.S. Provisional Application No. 60/476,233, filed Jun. 6, 2003, U.S. Provisional Application No. 60/628,483, filed Jul. 1, 2003, U.S. Provisional Application No. 60/491,528, filed Aug. 1, 2003 and U.S. Provisional Application No. 60/569,646, filed May 11, 2004, of the instant title, which are herein incorporated by reference in their entirety.

### FIELD OF INVENTION

The present invention relates to the field of gene expression analysis, and to methods of improving amplification reactions used to study gene expression. In particular, the invention relates to methods of improving quantitative gene expression analysis by inhibiting the amplification or reverse transcription of transcript species that impede gene expression analysis or skew the relative gene expression profile of the sample.

### BACKGROUND OF THE INVENTION

Life is substantially informationally based and its genetic content controls the growth and reproduction of the organism. The amino acid sequences of polypeptides, which are critical features of all living systems, are encoded by the genetic material of the cell. Further, polynucleotide sequences are also involved in control and regulation of gene expression. It therefore follows that the determination of the make-up of this genetic information has achieved significant scientific importance.

Gene expression analysis tells researchers which genes are “turned on” or “turned off” in a particular cell or tissue sample. Expressed genes are one component that determines which proteins in the cell are synthesized and to what extent. Specific expression patterns determine the cell type, as well as physiological conditions within the cell, including disease. Understanding changes in gene expression provides researchers with evidence of which genes and proteins play a role in a specific disease or physiological state, and can provide clues regarding genetic abnormalities, disease pathways, disease mechanisms of action and mechanisms of toxicity.

Whole blood is a particularly convenient sample for analyzing gene expression data. Removal of red blood cells (RBC) from whole blood samples, with subsequent purification and analysis of white blood cells (WBC) with regard to gene expression has produced the most useful data, despite the inconvenience and difficulties associated with such preparation. Indeed, while there are protocols that allow for the isolation of WBC from whole blood, these are potentially problematic due to the technical expertise and time required to rapidly isolate the cells which is less than ideal for most accrual sites. Also, if the cells are not processed in a short period of time, there is the potential for gene activation, which can make accurate monitoring of in vivo responses difficult. Due to these issues, a protocol that allows for comprehensive gene expression from whole blood would be useful.

Some commercial approaches claim to provide stabilization of whole blood in such a way that gene expression data is improved (see Rainen et al., 2002, Stabilization of mRNA in whole blood samples, Clin. Chem. 48(11): 1883-90). According to Rainen et al., accurate quantification of mRNA in whole blood is made difficult by the simultaneous degradation of gene transcripts and unintended gene induction caused by sample handling or uncontrolled activation of coagulation. The present inventors have found, however, that there are detectable genes in peripheral white blood cells that are not detected in samples of RNA isolated directly from whole blood when analyzed using commercial gene expression microarray technology. For this reason, the use of whole blood isolation and stabilizing protocols (i.e., Trizol, GITC) do not solve the gene expression analysis problems associated with whole blood.

### SUMMARY OF THE INVENTION

The present invention solves the gene expression analysis problems associated with existing methods of whole blood gene expression analysis by providing an improved method of analyzing gene expression in a cell or tissue sample wherein one or more transcripts, or representatives thereof, that skew the relative gene expression profile of the cell or tissue sample are removed or substantially inhibited or inactivated, prior to, during or subsequently to a reverse transcription reaction. In one embodiment, among others, a method of inhibiting amplification of one or more red blood cell mRNA transcript species in a sample that impede gene expression analysis of other transcript species in the sample is provided, comprising (a) adding one or more red blood cell nucleic acid sequence-specific interfering molecules to the sample; and (b) amplifying said transcript species in the sample in the presence of said one or more red blood cell nucleic acid sequence-specific interfering molecules. The invention also provides methods of identifying mRNA transcript species that skew the relative gene expression profile of the cell or tissue sample, and compositions and kits comprising interfering molecules that target such mRNA transcripts.

### BRIEF DESCRIPTION OF THE DRAWINGS

FIG. 1. Photograph of agarose gel depicting prominent band of approximately 600 base pairs in cRNA obtained from white blood cells versus whole blood.

FIG. 2. Illustration of the mechanism by which a gene specific primer of the invention blocks transcription by reverse transcriptase of a selected mRNA sequence.

### Definitions

In the context of the methods of the present invention, the term “amplification” should be construed as including any known amplification procedure, such as polymerase chain reaction (PCR), Nucleic Acid Sequence Based Amplification (NASBA), ligase chain reaction (LCR), strand displacement amplification (SDA), linear amplification strategies, in vitro transcription (IVT), i.e., of cDNA to form multiple cRNA transcripts, etc. It should be understood that while an amplification protocol as used herein may include a reverse transcription step, for instance where an mRNA molecule is first reverse transcribed into a cDNA molecule and the cDNA is then used to make multiple copies of the cDNA or cRNA via PCR or in vitro transcription, reverse transcription alone does not result in amplification of RNA species.

“Gene expression analysis” involves preparing and analyzing a population of mRNA transcripts, i.e., from a cell or tissue sample, in order to determine which genes are expressed in the sample. A typical gene expression analysis protocol involves reverse transcribing mRNA transcripts into cDNA molecules (an “RT” step), and then generating multiple “cRNA” transcripts from the cDNA via in vitro transcription using T7 RNA polymerase or another suitable RNA polymerase (an “IVT” step). “Quantitative gene expression analysis” includes, but is not limited to, analyses where a known quantity of endogenous or exogenous control sequence added to the reaction is simultaneously co-amplified to provide an internal standard for calibration, in order to determine the relative quantity of expression of the genes in the sample.

A “gene expression profile” provides the results of a gene expression analysis, and indicates some measure of the gene expression levels for at least one transcript found in a sample. Profiles also include analysis in which genes are detected in the sample being analyzed and/or not detected in the sample being analyzed. Although any platform technology may be used to produce gene expression profiles, microarray platforms such as those available from Affymetrix (Santa Clara, Calif. USA) may be a preferred technology.

Affymetrix defines present (i.e., detected) and absent (i.e., not detected) gene expression profiles in terms of present and absent calls. According to Affymetrix's “Statistical Algorithms Reference Guide”, each probe pair in a probe set is considered as having a potential “vote” in determining whether the measured transcript is detected (present) or not detected (absent). A probe pair is two probe cells designed as a Perfect Match (PM) and its corresponding Mismatch (MM), whereas a probe set is a collection of 11-20 probe pairs designed to detect a specific target sequence. A value called the discrimination score describes the vote. The discrimination score is calculated for each probe pair and is compared to a predefined threshold. Probe pairs with scores higher than the threshold vote for the presence of the transcript. Probe pairs with scores lower than the threshold vote for the absence of the transcript. The voting result is summarized as the p-value. The higher the discrimination scores are above the threshold, the smaller the p-value and the more likely the transcript will be present. The reverse is true for the lower the discrimination score. Affymetrix GeneChip® arrays are used with Affymetrix MAS 5.0 software to determine the present and absent calls.

Commercial nucleic acid arrays, such as Affymetrix's GeneChip® arrays, are commonly used to determine the percent and identity of detectable genes in a population via hybridization of the amplified cRNA transcripts or cDNA to an ordered array of different oligonucleotide probes that have been coupled to the surface of a solid substrate in different known locations. cRNA is an antisense RNA transcribed from a cDNA template. The transcripts are typically labeled during amplification to facilitate detection on the array. Such arrays have been generally described in the art, for example in U.S. Pat. No. 5,143,854, WO 90/15070 and WO 92/10092, each of which is herein incorporated by reference in its entirety. After hybridization and scanning of the array, the hybridization data is analyzed to identify which of the transcripts are present in the sample, as determined from the probes to which the labeled transcripts hybridized. Further, the fluorescence levels of each present gene can be identified and those levels used to produce comparative quantitative levels of gene expression. A variation of this procedure is using probes attached to multiple solid surfaces (i.e., Luminex, Illumina, bDNA) or suspended in solutions (Aclara).

The perfect match (PM) and mismatch (MM) probe set values are metrics that can be used to determine the accuracy of gene expression data. Mismatch control probes are identical to their perfect match partners except for a single base difference in a central position. The MM probes act as specificity controls that allow the direct subtraction of both background and cross-hybridization signals, and allow discrimination between “real” signals and those resulting from non-specific or semi-specific hybridization. Hybridization of the intended RNA molecules produces more signal for the PM probes than for the MM probes, resulting in consistent patterns that are highly unlikely to occur by chance. In the presence of even low concentrations of RNA, hybridization of the PM/MM probe pairs produces recognizable and quantitative fluorescent patterns. The strength of these patterns directly relates to the concentration of the RNA molecules in the complex sample. Thus, PM/MM probe sets allow one to determine whether a signal is generated by hybridization of the intended RNA molecule. When the signal from the MM probes is greater than that of the PM probes, non-specific or cross-hybridization is occurring. Samples with a high number of probe pairs with MM signals greater than PM signals usually are the result of poor quality sample preparation or hybridization and have poor quality expression data.

An unwanted or undesirable transcript according to the present invention is one whose presence “skews” the relative gene expression profile of the cell or tissue sample being studied. A transcript “skews” a relative gene expression profile when there is a decrease in detectable other transcript species when the transcript is included in the amplified sample as compared to when the transcript is either deleted or its amplification is inhibited. A transcript also skews a relative gene expression profile when its presence results in significantly decreased PM/MM ratios such that array analysis of the sample produces poor quality expression data. On arrays, the signal intensities for genes that skew the relative gene expression profile may be in the tens of thousands, as compared for instance to a signal intensity of about 20 for a gene that is not expressed (i.e., background), or a signal of about 100 for a gene showing a significant level of expression. By further comparison, the signals for beta actin and GADPH, which are control genes on the Affymetrix Gene Chip®, are in the 5000 range and are considered to be highly expressed.

An “interfering molecule” as used in the present invention is one that interferes or enables the user to interfere in any aspect with the final presence of one or more unwanted or undesirable transcript species in an amplified population. Accordingly, “inhibition” of amplification as it is used in the present invention refers to any means that results in deletion or reduction of the unwanted transcript or transcripts from the population of detectable transcripts. Such inhibition may occur at any stage of the amplification procedure, for instance by interfering with reverse transcription of the transcript or IVT or PCR of the corresponding cDNA, or by facilitating removal of the corresponding cRNA species prior to array hybridization analysis, for instance by the use of magnetic beads or cleavage or degradation. The interfering molecule may be RNA or DNA or a modified species of RNA or DNA. Such inhibition may be used to achieve an “improved” gene expression profile, i.e., where the number of detectable transcripts obtained is higher than the number of detectable transcripts obtained when amplification, and particularly reverse transcription, of the unwanted transcript is not inhibited.

An “interfering molecule” according to the invention is “specific” to the unwanted or undesirable transcript species being targeted. In this regard, “specific” means that the molecule is able to bind to or interact with the unwanted target transcript species or the complement thereof, for instance a cDNA strand corresponding thereto, with specificity. Binding or interacting “with specificity” means that the interfering molecule binds to or interacts with the targeted transcript species or a complement thereof and not substantially to other transcript species. Accordingly, for antisense interfering molecules, such molecules are generally at least about 90% identical in sequence to the complementary strand of the targeted transcript species in order to provide binding specificity. However, it should be noted that the position at which the Watson-Crick base pairing is disrupted is very important as are the hybridization conditions. The position of the disrupted base pairing is important to determine the degree of duplex destabilization. Incorrect base pairing at the ends of a duplex are less destabilizing than incorrect base pairing in the middle of the duplex.

A “reverse transcriptase” according to the invention is any reverse transcriptase enzyme known in the art that may be used in an in vitro reverse transcription reaction, including but not limited to AMV, MMLV, HIV, FIV, Telomerase, and rTth. AMV and MMLV can be RNase H negative or positive. Telomerase is described as a reverse transcriptase by Cech et al., The Telomere and Telomerase: Nucleic Acid—Protein Complexes Acting in a Telomere Homeostasis System: A Review. (1997) Biochemistry (Mosc), 62, 1202-1205. A “RNA polymerase” according to the invention is any RNA polymerase enzyme known in the art that may be used to facilitate an in vitro transcription reaction, including but not limited to T7, T3, SP6 or modified versions (i.e. to increase processivity), and RNA pol II. Any other enzyme known in the art and useful for performing the desired amplification reaction may also be used, including thermostable DNA polymerases, ligase enzymes, etc.

A “whole blood” sample according to the invention may comprise a number of cell types, including but not limited to red blood cells (RBC), white blood cells (WBC), platelets, etc. There are five types of WBC that total in the thousands per microliter of blood: the granulocytes (in order of abundance: neutrophils, eosinophils, and basophils) and the mononuclear cells (lymphocytes and monocytes). There are about 5 million RBC and 300,000 platelets per microliter of blood. Within the RBC population, about 1% are reticulocytes that are actively making mRNA. A reticulocyte is an immature red blood cell which has extruded its nucleus. Reticulocytes contain large amounts of RNA and ribosomes which are gradually lost over the two day period it takes the reticulocyte to mature into an erythrocyte. Reticulocytes use the RNA to produce hemoglobin, the synthesis of which comes to a halt once RNA is depleted. The hemoglobin produced by the reticulocytes is thus the hemoglobin present in the mature erythrocytes. Reticulocytes spend one day in the bone and one day in the blood. While in the blood, reticulocytes are only distinguishable from mature erythrocytes using special supravital stains. Erythrocytes are mature red blood cells that circulate in the bloodstream for about 120 days before being destroyed by the reticuloendothelial system.

The methods of the invention are particularly useful for whole blood total RNA analyses, where it is difficult to remove RBC prior to RNA analysis, and where removal of such cells would remove a portion of the biologically relevant data. However, the methods will also find use in gene expression analysis of any tissue that contains erythrocytes, including but not limited to tissues selected from the group consisting of spleen, bone marrow, placenta, vascularized tumor, angioid tumor, adipose, lung, muscle, pancreas, heart, brain, liver and hemorrhagic tissues.

### DETAILED DESCRIPTION OF THE INVENTION

The present invention concerns methods of improving gene expression analysis of a cell or tissue sample containing one or more unwanted gene transcripts that are shown to skew the gene expression profile of the cell or tissue sample. In addition, such methods comprise identifying such undesirable transcripts in a given sample population. In particular, the inventors have identified transcripts in whole blood and erythrocyte-containing tissues that skew the relative gene expression profile obtained from such samples, particularly profile analyses performed on microarray platforms like the GeneChip® array, CodeLink™, and others.

For instance, the present inventors have discovered that without the improvements of the present invention, red blood cell RNA, for instance, globin RNA, in peripheral blood that has been copurified during total RNA isolation from whole blood samples interferes with the correct determination of the cRNA to be loaded on GeneChip® arrays and increases cross hybridization. This interference results in lower general present calls and lower numbers of detectable genes, and consequently an inaccurate determination of gene expression values from whole blood samples.

To illustrate, blood samples that have been processed to remove red blood cells (RBC) typically show approximately 40% present calls (˜9,000 out of ˜22,000 genes on Affymetrix's HuU133A GeneChip® array). On the other hand, samples processed from whole blood exhibited a decrease in the total number of genes called present (˜5,000 out of ˜22,000 genes or ˜24%). Between the two preparations, the overlap of whole blood to WBC detectable genes is ˜90%. Thus, while there are fewer expressed genes detected (with proportionally fewer representatives from each known cell type), the data from whole blood is biologically relevant (meaning removal of RBC prior to RNA isolation is not an ideal solution). Further, in addition to the decreased number of detectable genes, there is an increase in the number of probe pairs where the signal from the mismatch is greater than that from the perfect match (i.e. increase MM/PM ratio). Because the ratio of mismatched to perfect match probe pairs is a quality control metric, the microarray chips fail QC.

The present inventors have also observed that the mass amount of reticulocyte RNA in whole blood total RNA preparations results in a visible, dominant RNA species or group of species in both the mRNA preparation and the resulting IVT cRNA sample. This species or group of transcripts is visible as a dominant band of about 600 base pairs when mRNA samples and cRNA preparations are observed on an agarose gel. The inventors have surprisingly discovered that this dominant band contains hemoglobin transcripts from red blood cell mRNA, and that when amplification of globin RNAs is blocked during reverse transcription, a concomitant increase in the number of general detectable gene and gene expression values is achieved.

Thus, the present invention includes methods of identifying undesirable transcripts in cell or tissue samples that skew the relative gene expression profile when co-amplified with the other transcripts in the population. The invention also includes methods of inhibiting amplification of one or more of such undesirable transcript species in a sample, by removing the one or more transcripts that skew the relative gene expression profile prior to or during an amplification or reverse transcription reaction. The invention further includes methods of improving or enhancing gene expression analysis of a sample containing one or more undesirable transcript species, wherein the improvement comprises removing or inhibiting the amplification of undesirable transcript species and thereby achieving an increase in the number of detectable genes than would have been obtained in the presence of the undesirable transcript species.

While the methods of the invention may be used to improve the gene expression analysis of any sample containing one or more such unwanted transcripts, the methods are especially useful for removing, or removing the effect of, unwanted transcripts from whole blood and erythrocyte-containing tissues. In addition, the methods are applicable to any type of amplification reaction of a mixed sample of nucleic acids, where one or more individual nucleic acids in the population are present to such an extent that the amplification of such transcripts impedes the analysis of the remaining population.

Where the methods of the invention are used for the analysis of whole blood or erythrocyte-containing tissues, the inhibition process comprises (a) adding one or more red blood cell nucleic acid sequence-specific interfering molecules to the sample; and (b) amplifying transcript species in the sample in the presence of said one or more red blood cell nucleic acid sequence-specific interfering molecules. In particular, the present invention comprises methods for inhibiting amplification of red blood cell specific genes, for example one or more globin mRNA molecules, in a sample containing RNA during a nucleic acid amplification process, comprising (a) adding one or more globin nucleic acid sequence-specific interfering molecules to the sample; and (b) amplifying said RNA in the sample in the presence of said one or more globin nucleic acid sequence-specific interfering molecules.

Any type of gene expression analysis measuring more than one gene simultaneously will benefit from the methods of the invention, particularly “quantitative” analyses, for instance, those methods where a known quantity of control sequence is simultaneously co-amplified to provide an internal standard for calibration. Gene expression analysis may be performed using a variety of amplification reactions, for instance by reverse transcription of mRNA in the sample into cDNA, and further, optionally synthesizing cRNA transcripts from each cDNA molecule using an RNA polymerase. Alternatively, gene expression analyses may include a step wherein further cDNA molecules are synthesized using DNA polymerase, for instance as in PCR or other known amplification reactions.

When microarray technologies are used to monitor gene expression, mRNA molecules are often converted to cDNA through the use of reverse transcriptases to a cDNA molecule and then to a double stranded cDNA molecule through the use of polymerases. The cDNA molecules are then used to generate multiple antisense or cRNA copies of the cDNA through the activity of various RNA polymerases. During this final amplification process, modified nucleotides are incorporated in the reaction mixtures, and hence into the cRNA molecules. These modified nucleotides are then used to generate a detectable signal through the interaction with other molecules that either contain a signal or can generate a signal. The labeled cRNA is then reacted with the probes on the array, where the cRNA hybridizes to the gene specific probes on the array.

In such amplification reactions, inhibition of amplification may occur at any step during the amplification process, including at the step of reverse transcription of mRNA into cDNA, or at the step of cRNA or cDNA synthesis from cDNA with RNA or DNA polymerase, respectively. Inhibition of amplification may also occur by deleting the original unwanted red blood cell mRNA species or the resulting cRNA species prior to analysis, for instance by cleavage or degradation as described in more detail below, or by the use of magnetic particles attached to complementary oligonucleotides. Thus, as defined above, an “interfering molecule” as used in the present invention is one that interferes in any aspect with the final presence of one or more target red blood cell transcript species in a sample, rather than a molecule that only interferes with a reverse transcriptase or polymerase reaction.

To interfere with the enzymatic reactions in the amplification process, it is possible to design a number of nucleic acid molecules that can act via a blocking antisense mechanism (physical barrier to enzymatic processing of mRNA or cDNA by various polymerase enzymes) or via a triple stranded (Hoogstein base paired) mechanism. It is also possible to inhibit enzymatic reading of the mRNA or cDNA molecules using a sequence specific oligonucleotide that has a cross linking functional group (psoralen, etc.).

Additionally, it is possible to specifically degrade the unwanted mRNA(s) in the total RNA pool or the resulting cRNAs by using antisense oligonucleotides that invoke RNase H mediated cleavage of the targeted red blood cell mRNA, or via an antisense oligomer that uses a catalytic functional group (EDTA, etc.) that can mediate the degradation of the unwanted target mRNA(s).

Thus, to interfere with transcriptase or polymerase reactions, one can use unmodified DNA antisense oligonucleotides. Such oligonucleotides support RNase H activity, but the operator may see increased degradation of non-targeted mRNA due to potential for sufficient transient hybridization events that allow for RNase H to cleave the RNA component of the heteroduplex. Alternatively, unmodified antisense DNA or RNA oligonucleotides may be used as blocking molecules, although adding blocking modifications as further described below to the 5′ or 3′ end depending on the amplification step to be inhibited is advantageous to prevent elongation from the antisense oligonucleotide.

It is also possible to use chimeric oligonucleotides that have a portion comprised of modifications that do not support RNase H activity, such that when they hybridize to non-target RNA species the ability to support RNase H activity is minimized. Thus the potential for non-target mRNAs to be inadvertently cleaved by RNase H is reduced and the overall integrity of the mRNA pool is maintained. This should minimize the number of sequences that can support RNase H activity, so the overall integrity of the mRNA will be of higher quality than if an unmodified DNA oligomer was employed. Suitable modifications include but are not limited to sugar modifications (2′O-alkyl modifications such as 2′O-methyl, 2′O-butyl, and 2′O-propyl; 2′-O-halide modifications such as 2′O—F and 2′O—Br; and 2′O-methoxyethoxy,), carbocyclic (non-Oxygen) sugar mimics, bicyclic sugars (alkyl bridged between 1′ and 3′ positions or 1′ and 4′ positions, etc.) modifications to the backbone (PNAs, 2′-5′ linked oligomers, alpha-linked oligomers, borano-phosphate modified oligomers, chimeric oligomers, including anionic, cationic and neutral backbone structures, etc), or modifications to the phosphodiester backbone (phosphorothioate, diphosphorotiooate, phosphoroamidate, methylphosphonate, etc.).

It is also possible to use modified oligonucleotides that do not necessarily support RNase H activity but bind with sufficient strength to prevent polymerases and transcriptases from being able to transcribe or reverse transcribe (i.e. “read through”) the oligomers, acting as a physical block to nucleic acid duplication. Reverse transcriptases, polymerases, and other protein(s) have the ability to “melt” through secondary structures (duplex structures) in nucleic acids and thus may be able to “read through” the blocking oligomer and complete making the reverse complementary nucleic acid to the template nucleic acid. By using modifications that increase the binding affinity of the oligomer to the targeted mRNA, it is possible to inhibit polymerases and transcriptases that are copying the template nucleic acid and prevent the faithful replication by aborting the enzyme's ability to “read through” the duplex structure formed by the oligomer and the target mRNA. Modifications can include, but are not limited to, 2′O-alkyl, 2′O—F, PNA, and 5 methyl C substitutions.

It is also possible to use antisense oligomers that have attached to them a functional RNase H moiety that will cleave the RNA, and prevent faithful copying by enzymatic methods. In this instance, the RNase H moiety will fold back on the heteroduplex and cleave the RNA component. This approach also provides an advantage in that by locking down the activity of the RNase H onto the oligonucleotide, the potential for spurious cleavage of non-target RNA is reduced since the hybrid is limited to the ability to cleave at a specified distance that is defined by the length of the linker between the RNase H and the oligomer. Catalytic ribozymes can be also used to target the mRNA or cRNA and elicit the cleavage, so long as the sequence requirements for ribozyme activity are present in the target RNA.

It is also possible to use antisense oligomers that have an attached functional moiety that will cleave the RNA after activation, and prevent faithful copying by enzymatic methods. Such functional moieties are activated to form a chemical bond with the RNA component. Certain chemistries that can be used include but are not limited to aldolating agents, alkylating agents, psoralen or EDTA. Activating agents can include ultraviolet light, ferric/ferrous ionic compounds, etc. By attaching the functional moiety to the oligonucleotide by a linker the potential for spurious chemical attachment to non-target RNA is reduced since the activity is limited to the formation of the heteroduplex at the end nearest the moiety, such that the moiety is in close spatial proximity to the target RNA. The ability of the moiety to “attack” the target mRNA is dependent upon this proximity.

Non-antisense strategies for inhibiting amplification are also included in the methods of the invention. For instance, triple stranded oligomers may be formed at areas of purine or pyrimidine stretches in the mRNA via Hoogstein base pairing that act as a physical block to polymerases and reverse transcriptases. Triple strands may be mediated by two separate oligomers that are component sequences that allow for triplex formation. Also, it is possible to use circular nucleic acids, or dumbbell, or stem-loop structures that have within their sequence the necessary two sequences, located opposite each other in the circle or stems, that support triplex formation. For these structures, the loop size of the non-triplex forming sequences should be sufficiently long to allow for such structures to form, but not too long to prevent the two triplex forming sequences from being in close proximity to associate with the mRNA sequence.

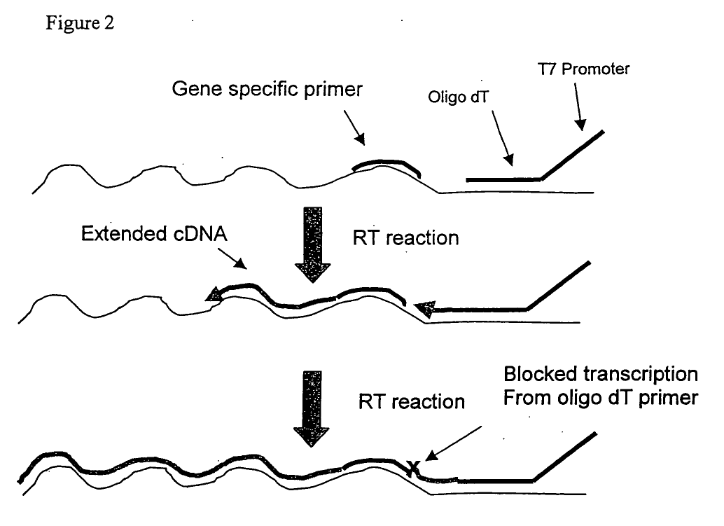

In some embodiments of the invention, gene-specific primers are designed and used to interfere with the enzymatic reactions in the amplification process. For example, it is possible to inhibit enzymatic reading of the mRNA molecules during cDNA synthesis by using a selected gene-specific primer that binds to the mRNA whose replication is to be suppressed, e.g., human or other mammalian globin mRNA. The gene-specific primer binds downstream of the transcription initiation primer (typically a poly-dT T7 or T3 promoter-containing primer). In the presence of reverse transcriptase, the gene-specific primer is extended in the 3′ direction, as is the transcription initiation primer, but transcription from this primer is halted when this cDNA approaches the block created by the gene-specific primer. The block serves to inhibit translocation of the reverse transcriptase. Thus, cDNA containing a promoter region is not produced, thereby preventing replication of cDNA or cRNA corresponding to the selected gene when DNA polymerase or RNA polymerase is added to a sample. FIG. 2 illustrates how synthesis of cDNA by reverse transcriptase is inhibited by a gene-specific primer of the invention.

In some embodiments of the invention, the gene-specific primer is designed to contain a relatively higher number of G and C residues at its 5′ end to increase the binding affinity of the primer and prevent dissociation or “melting off” in subsequent reactions. The invention also contemplates the use of chimeric gene-specific primers, as long as these primers support chain elongation by reverse transcriptase. The longer the extension from the gene-specific primer is, the more stable the resulting heteroduplex is, which further impairs the ability of reverse transcriptase to extend the cDNA from the oligo-dT primer.

The methods of the invention stand in contrast to the use of an oligomer that cannot act as a primer, for example, one blocked at a 3-OH position with a phosphate or other blocking group, or one with substituents such as a ribose O-methyl group or modified phosphate backbone, as discussed above.

The present invention is the first to the inventors' knowledge to identify transcripts whose presence skews the relative gene expression of a sample according to the parameters defined herein. Accordingly, the present invention also encompasses kits and compositions containing interfering molecules that target such transcripts as identified herein. Methods of identifying undesirable transcripts include identifying the sequence or sequences of dominant transcripts in an RNA sample, for instance as viewed on an agarose or acrylamide gel, or identifying species in an amplified population that have signal intensities in the tens of thousands when analyzed on a GeneChip® or other gene expression array. Other methods of identifying such undesirable transcripts will be apparent to one of skill in the art depending on the cell or tissue sample being analyzed.

Exemplary Target Genes and Interfering Molecules

The methods of the invention may be used to improve gene expression analyses from any species of plant or animal, vertebrate or invertebrate, fungi, bacteria, etc. For instance, the methods of the invention may be used to improve the analysis of gene expression in animal species including but not limited to human, rat, murine, rabbit, guinea pig, dog, cat, primate, equine, bovine, porcine, ovine and chicken. The sequences of the globin genes in various species are known and may be used to design interfering molecules according to the present invention. Globin interfering molecules can be DNA or RNA. For instance, suitable RNA interfering molecules for inhibiting amplification of human, rat and canine globin mRNAs may contain or comprise sequences such as the following (note that the “U”s become “T”s for corresponding DNA interfering molecules, and that sequences are shown in 5′ to 3′ order):

Human beta globin 01 (SEQ ID No. 1)
GCAGAAUCCAGAUGCUCAAG
Human beta globin 02 (SEQ ID No. 2)
GGACAGCAAGAAAGCGAGCUUUG
Human beta globin 03 (SEQ ID No. 3)
CAUUGAGCCACACCAGCCACC
Human alpha globin 04 (SEQ ID No. 4)
UUUGCCGCCCACUCAGACUU
Human alpha globin 05 (SEQ ID No. 5)
CCACCGAGGCUCCAGCUUAACGG
Human alpha globin 06 (SEQ ID No. 6)
GUCCACCCGAAGCUUGUGCGCGU
Human gamma globin 07 (SEQ ID No. 7)
UGUGAUCUCUCAGCAGAAUAGAU
Human gamma globin 08 (SEQ ID No. 8)
GCCUAUCCUUGAAAGCUCUGAAU
Human gamma globin 09 (SEQ ID No. 9)
CCACUGCAGUCACCAUCUUCUGC
Rat beta globin 01 (SEQ ID No. 10)
GCAGUGAAAGUAAAUGCCUU
Rat beta globin 02 (SEQ ID No. 11)
GACAACAACUGACAGAUGCUCUC
Rat beta globin 03 (SEQ ID No. 12)
CCACCUUCUGGAAGGCAGCCUGUGC
Rat beta globin 04 (SEQ ID No. 13)
GCUCUCUUGGGAACAAUUGACC
Rat beta globin 05 (SEQ ID No. 14)
GGCACUGGCCACUCCAGCCACC
Rat beta globin 06 (SEQ ID No. 15)
CCAGGAGCCUGAAGUUCUCAG
Rat alpha globin 07 (SEQ ID No. 16)
UUGCUUCCUACUCAGGCUU
Rat alpha globin 08 (SEQ ID No. 17)
AGAGGUAUAGGUGCAAGGGAGG
Rat alpha globin 09 (SEQ ID No. 18)
GGUCAGCACAGUGCUCACAGAG
Human beta globin 10 (SEQ ID No. 19)
GCAUUAGCCACACCAGCCACCAC
Human delta globin 11 (SEQ ID No. 20)
UGAAGUUGAGCUGAACAUUCUUUAU
Human delta globin 12 (SEQ ID No. 21)
GCAGAAGCCAUACCCUUGAAGUAG
Human delta globin 13 (SEQ ID No. 22)
GUGUUCCCAAGUUCAGAAAAUAG
Human delta globin 14 (SEQ ID No. 23)
GUUAUCAGGAAACAGUCCAGGAUCUC
Canine alpha globin 1 (SEQ ID No. 24)
GCGAAGAACUUGUCCAGGUAGGCG
Canine beta globin 2 (SEQ ID No. 25)
CUUCCAGUGGUCACCAGGAAACAG
Rat alpha globin 10 (SEQ ID No. 45)
GGACGGAAGAAGGGCCUGGUCAG
Rat alpha globin 11 (SEQ ID No. 46)
GCAAGCCCGACAGGAGGUGGCU
Rat alpha globin 12 (SEQ ID No. 47)
CAGAGUCUUCUUUCCUAGUUCUGC
Rat alpha globin 13 (SEQ ID No. 48)
CUUUGCACAUGCAUAUAAAUAG
Rat alpha globin 14 (SEQ ID No. 49)
UUAUUCAAAUACUGGUUCAG

Other suitable sequences are disclosed throughout the application, for instance in the examples section.

While it is particularly advantageous to inhibit the amplification of globin RNA sequences during gene expression analyses of erythrocyte-containing tissues, including alpha (HBA1, HBA2), beta (HBB), gamma (HBG1, HBG2), delta (HBD), epsilon (HBE1), theta (HBQ1), and zeta (HBZ) globin sequences and variants thereof, other red blood cell RNA transcript species that impede gene expression analysis may also be targeted either singularly or in combination with any of the globin transcript species, including but not limited to transcripts for ribosomal proteins L3 (RPL3L), L6 (RPL6), L7 (RPL7), L7a (RPL7A), L9 (RPL9), L10a (RPL10A), L11 (RPL11), L12 (RPL12), L13a) RPL13A), L17 (RPL17), L18 (RPL18), L19 (RPL19), L21, L23a (RPL23A), L24 (RPL24), L27 (RPL27), L27a (RPL27A), L28 (RPL28), L30 (RPL30), L31 (RPL31), L32 (RPL32), L34 (RPL34), L35 (RPL35), L37 (RPL37), L37a (RPL37A), L41 (RPL41), S2 (RPS2), S3a (RPS3A), S5 (RPS5), S6 (RPS6), S7 (RPS7), S10 (RPS10), S11 (RPS11), S13 (RPS13), S16 (RPS16), S17 (RPS17), S18 (RPS18), S23 (RPS23), S24 (RPS24), S27a (RPS27A), S31 (RPS31), SM, large ribosomal protein PO(RPLPO), flavin reductase (BLVRB), ferrochelatase (FECH), myosin light protein (MYL4), synucleic alpha (SNCA), delta-aminolevulinate synthetase 2 (ALSA2), selenium binding protein 1 (SELENBP1), erythrocyte membrane protein bands 4.2 (EPB42) and 4.9 (EPB49), glycophorin C (GYPC), antioxidant protein 2 (AOP2), beta actin (ACTB), gamma actin 1 (ACTG1), vimentin (VIM), adipocyte fatty acid binding protein 4 (FABP4), eukaryotic translation elongation factor 1 alpha 1 (EEF1E1), translationally-controlled 1 tumor protein (TPT1), ubiquitin C (UBC), ferritin light polypeptide (FTL), leukocyte receptor cluster (LRC) member 7 (LENG7), beta-2-microglobulin (B2M), glyceraldehyde-3-phosphate dehydrogenase (GAPD), replication factor C (activator 1) (RFC1), heterogeneous nuclear ribonucleoprotein A1 (HNRPR1), Finkel-Bis kis-Reilly murine sarcoma virus (FBR-MuSV) ubiquitously expressed (fox derived) (FAU), ras homolog gene family member A (ARHA), cofilin 1 (non-muscle) (CFL1), ornithine decarboxylase antizyme 1 (OAZ1), microsomal glutathione S-transferase 1 (MGST1), early growth response 1 (EGR1), microsomal glutathione S-transferase 1 (MGST1), peptidylprolyl isomerase A (cyclophilin A) (PPIA), carcinoembryonic antigen-related cell adhesion molecule 5 (CEACAM5), galactoside-binding lectin 4 (LGALS4), liver fatty acid binding protein 1 ((FABP1), coatomer protein complex subunit gamma (immunoglobulin lambda joining 3) (COPG, IGLJ3), major histocompatibility complex class 1B (HLA-B), major histocompatibility complex class 1C (HLA-C), immunoglobulin heavy mu constant (IGHM), immunoglobulin kappa constant (IGKC), solute carrier family 25 member 3 (SLC25A3), H3 histone family 3A (H₃FA), normal mucosa of esophagus specific 1 (NMES1), heat shock 70 kDa protein 8 (HSPA8), hypothetical protein MGC14697 (MGC14697), polymeric immunoglobulin receptor (PIGR), and FK 506 binding protein 8 (FKBP8), hypothetical protein BC012775 (LOC91300), cold shock domain protein A (CSDA), F-box only protein 7 (FBX07), CGI-45 protein (CGI-45) makorin ring finger protein 1 (MKRN1), small EDRK-rich factor 2 (SERF2), pinin (PNN), SET domain bifurcated 1 anti-oxidant protein 2 (AOP2, SETDB1), nuclease sensitive element binding protein (NSEP1), glutathione peroxidase 1 (GPX1), MAX interacting protein 1 (MXI1), and ubiquitin B (UBB). Suitable interfering molecules for inhibiting FK 506 binding protein 8 AND selenium binding protein 1 are:

FK 506 Binding Protein 8 (01) (SEQ ID No. 26)
GAAGGGCUGCCCCCAGGCCUGUUGAG
FK 506 Binding Protein 8 (02) (SEQ ID No. 27)
GAGGCCAGCCCUGGCGGAGACCUAGCCCA
FK 506 Binding Protein 8 (03) (SEQ ID No. 28)
CCUCUGGGCUUUCCUCCUAGAGG
FK 506 Binding Protein 9 (04) (SEQ ID No. 29)
CCUGCUGGCUGGGCUGCACGACCC
Selenium Binding Protein 1 (01) (SEQ ID No. 30)
CAGCACAGUGAGCAACAAGCAAC
Selenium Binding Protein 1 (02) (SEQ ID No. 31)
CUUGGUGCCUCCAAGAGCUGCCAAG
Selenium Binding Protein 1 (03) (SEQ ID No. 32)
CAAGAGAGAGCAGAAUGAAGCCAG
Selenium Binding Protein 1 (04) (SEQ ID No. 33)
GUGAUGAGGGUGGAGUUCAAAUC

Applications

The methods of the invention may be used in any application where one or more nucleic acid species skews or impedes analysis of an amplification reaction of a mixed population. For instance, as mentioned above, the methods of the invention may be used in performing quantitative gene expression analysis using GeneChip® or other arrays. The method of the invention may be used in screening humans for the presence of disease marker for susceptibility to specific diseases. The methods of the invention may also be used in analyzing animal blood or tissue samples, for instance in Gene Logic's ToxExpress® system for analyzing the effects of potential toxic compounds on gene expression profiles. See application Ser. Nos. 09/917,800, 10/060,087, 10/191,803, 10/338,044, 10/357,507 and 60/395,355, which are herein incorporated by reference in its entirety.

### EXAMPLES

The following examples are provided to describe and illustrate the present invention. As such, they should not be construed to limit the scope of the invention. Those in the art will well appreciate that many other embodiments also fall within the scope of the invention, as it is described herein above and in the claims.

### Example 1

### Identification of Globin mRNA Molecules as Dominant Transcript Species in Whole Blood

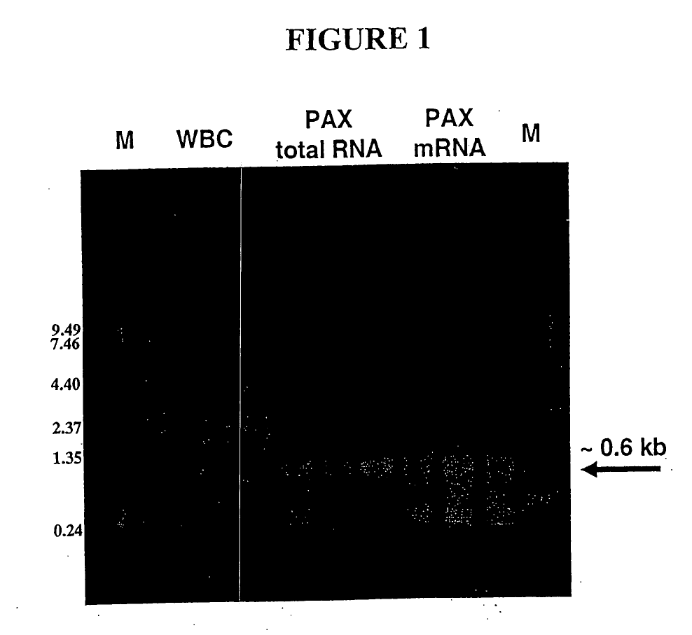

In processing whole blood samples (human, rat, mouse, etc.) for gene expression analysis, the present inventors observed that there is the potential for certain over expressed genes to impair the ability to monitor other genes that are expressed in the sample. For instance, in preparations of total RNA from whole blood, there appears to be at least one unique mRNA that is over expressed at a very high level. In a typical analysis of gene expression, the total RNA is amplified through a series of reactions to generate antisense RNA or cRNA that has incorporated into it modified nucleotides that allow for the generation of a signal that can be measured to determine the amount of cRNA generated for each original mRNA in the total RNA sample. When one conducts this amplification of total RNA isolated from whole blood, there is a large amount of cRNA(s) present in the cRNA pool that exhibits a size of approximately 600 nucleotides in length (see FIG. 1).

Experimental analysis of the whole blood preparations in comparison to whole blood preparations where the peripheral white blood cells have been removed shows that this over expressed cRNA(s) is still present. When cRNA from isolated peripheral white blood cells is examined, this over expressed cRNA band(s) is not present (see FIG. 1). Hence, the over expressed cRNA band(s) is derived from either erythrocytes or some other non-white blood cell components (for example, platelets).

Further analysis of the data revealed that there is a unique set of probes that correspond to globin genes (alpha, beta, and gamma) that exhibit higher levels of expression in whole blood cell preparations. In whole blood, these globin genes are expressed at high levels in red blood cells with gamma being found in fetal or new born individuals but decreasing upon aging and the alpha and beta forms being expressed at higher levels after birth. The length of the globin genes are known with alpha being −567 nucleotides, beta being −626 nucleotides, and gamma being −574 nucleotides long. Hence, in the cRNA, the presence of an amplified band around 600 nucleotides in length would indicate that this band(s) may be derived from one or more of these globin genes (the resolution of the electrophoresis gel is not sufficient to resolve the individual bands as the difference in their lengths is not large enough).

### Example 2

### Gene Expression Analysis of Whole Blood Samples With and Without Interfering Molecules

To date, samples of blood have been processed to remove red blood cells (RBC) so that the expression from the therapeutic and diagnostic relevant white blood cells (WBC) can be obtained. As described above, these samples typically show approximately 40% present calls (˜9,000 out of ˜22,000 genes on the Affymetrix GeneChip® Hu133A human array). Samples processed from whole blood exhibit a decrease in the total number of genes called present (˜5,000 out of ˜22,000 genes or ˜24%). In addition to the decreased present calls from whole blood samples, there is a increase in the number of probe pairs where the signal from the mismatch is greater than that from the perfect match (i.e. increase MM/PM ratio). As the number of mismatched probes whose signal is greater than that of the perfect matched probes increases, the quality of the gene expression data is compromised.

The amount of cRNA loaded onto the array was increased to compensate for the large amount of globin cRNA, to see if this permitted the monitoring of more genes. However, increasing the load of cRNA (up to 40 μg) did not result in a significant increase in the number of present calls (increased ˜4% from 24% to 28%) from whole blood, but did slightly decrease the MM/PM ratio that was causing the chips to fail QC. Further, using polyA-selected mRNA in place of total RNA increased the present calls to about 31% but did not reduce the high MM/PM ratio. Consequently, such preparations still exhibit compromised gene expression data.

The next experiment was to block primer-directed reverse transcriptase off the highly expressed globin mRNAs found in whole blood preparations. Three different blocking oligomers (“blockers”) were designed in the most 3′ region of alpha, beta and gamma globin. The oligomers were comprised of modified RNA nucleotides (2′O-methyl modified) to increase the stability of hybrid formation, and various lengths were tested to optimize the capacity to inhibit RT translocation. In some experiments, it was found that a combination of more than one oligomer per globin mRNA species produced maximum inhibition. With single blockers, there were full length and truncated bands produced (as seen via cRNA QC gel analysis), suggesting that the reverse transcriptase may not be completely inhibited. In general, longer blockers were more effective at inhibiting RT translocation than shorter blockers.

The Table below shows data where whole blood total RNA was evaluated using the Affymetrix HU133 GeneChip® array with and without nine blockers (three different blockers for each of alpha, beta and gamma globin). Briefly, to 1 μg of starting total RNA from whole blood, the blocker mixes at 0, 10 and 100 pmoles of each oligomer were added prior to first strand cDNA synthesis reaction and samples were subsequently processed to biotin labeled cRNA and processed according to Affymetrix SOPs for chip hybridization, washing, staining, and data capture.

The QC results (see Table 1) are from whole blood total RNA preparations in the absence (CTRL) or presence (two different concentrations) of nine blockers targeting alpha, beta, and gamma globin. Artificially produced cRNA transcripts to bacterial genes spiked in at the cRNA hybridization stage were unchanged in the presence of blockers. There was also no change in the log intensity/log background (i.e. signal to noise ratio). Note that there is a 15% decrease in the number of probe pairs where the signal from the mismatch is greater than that from the perfect match (i.e. MM/PM ratio) supporting an improvement in performance. The number of Li/Wong outliers was also reduced (“Model-based analysis of oligonucleotide arrays: expression index computation and outlier detection”, Li, C. and Wong, PNAS 98(1):31-36, 2001). The 5′/3′ ratios for GAPDH and B-Actin are also QC metrics. An increased ratio is indicative of a successful cDNA synthesis and an increase in the ability of the cRNA sample to react with chip probe sets that are designed to more 5′ regions. The 5′/3′ ratios for GAPDH and B-Actin were increased by 9% and 29% respectively with 100 pmole blockers. Also, the blockers showed a dose-dependent response on the percentage present calls. For the 10 pmole mix sample there was a 46% increase in percentage present and with the 100 pmole mix the gain was 61% when compared to unblocked whole blood samples. Gel analysis showed that in the presence of 100 pmole of each blocker, the dominant 600 base pair cRNA band was not evident.

TABLE 1

| | | Count of | | | | | | | |
|---|---|---|---|---|---|---|---|---|---|
| | | Negative PM- | | | | | | | |
| | log(Intens)/ | MM probe | Li/Wong | SpikeIn R- | SpikeIn | SpikeIn | Raw 5′/3′ | Raw 5′/3′ | |
| | log(BG) | pairs | Outliers | Squared | Intercept | Slope | GapDH | B-Actin | % Present |
| Untreated whole blood | 1.365 | 91140 | 3480 | 0.976 | 5.439 | 0.792 | 0.616 | 0.621 | 24.4 |
| RNA (CTRL) | | | | | | | | | |
| Whole blood RNA | 1.350 | 80811 | 2781 | 0.982 | 5.196 | 0.817 | 0.636 | 0.775 | 35.6 |
| treated with | | | | | | | | | |
| 10 pmole oligos | | | | | | | | | |
| Whole blood RNA | 1.360 | 77644 | 2435 | 0.982 | 5.382 | 0.81 | 0.671 | 0.799 | 39.3 |
| treated with | | | | | | | | | |
| 100 pmole oligos | | | | | | | | | |

To determine how the increased gene expression from whole blood correlates to that obtained from a WBC preparation, expression data from total RNA+Blockers (n=1 per each concentration) was compared to expression data derived from WBC preparation (see Table 2). Using the WBC data, a list of genes which were called Present in all 3 WBC preps was shown to contain 9662 out of 22283 total (about 43% Present). These genes were compared to those generated on the three whole blood preparations (CTRL, 10 pmoles, and 100 pmoles). Of the 5429 Present genes on the control chip, 4915 were in the WBC filtered list (90.5%). Of the 7939 Present genes on the 10 pmole mix chip, 7204 were in the WBC filtered list (90.7%). And of the 8763 Present genes on the 100 pmole mix chip, 7861 were in the WBC filtered list (89.7%). This shows that the present calls gained with the use of blockers were consistently (˜90%) found on the WBC gene list.

TABLE 2

| | | | | | | % of Whole |
|---|---|---|---|---|---|---|
| | | | | | | Blood P Calls |
| | | | | | % of Whole | Gained by |
| | Present | % of Total | P Calls in | % Overlap | Blood P Calls | Treat-ment In |
| | Calls | 22,283 | WBC | with WBC | in WBC | WBC |
| WBC | 9662 | 43 | 9662 | 100 | | |
| Whole | 5429 | 24 | 4915 | 51 | 91 | |
| Blood | | | | | | |
| (CTRL) | | | | | | |
| Whole | 7939 | 36 | 7204 | 75 | 91 | 91 |
| Blood + 10 pmoles | | | | | | |
| Whole | 8763 | 39 | 7861 | 81 | 90 | 88 |
| Blood + 100 pmoles | | | | | | |

This data shows that the use of blockers increases the WBC gene coverage in whole blood samples from 50.9% to 81.4% (from 4915 to 7861 out of 9662 possible), or that in essence, the modification to the protocol resulted in a recovery of an additional 30.5% of the total number of WBC expressed genes in whole blood (this represents a 60% increase over whole blood samples).

### Example 3

### Identification of Other Dominant Transcript Species in Whole Blood

When comparing those genes that are expressed in purified WBC and not in the whole blood RNA+blockers, only 28 fragments were identified. Of these there were only 3 which would appear to be characteristic of activated immune cells, mostly monocytes, and very slightly at that (II-1β, MHCII, and CD69). The rest were generally characteristically expressed by somewhat proliferative cells or hematopoietic cell types which one would expect to be enriched using this procedure over the whole blood preparation with blockers.

In looking at the genes that were expressed in whole blood +Blockers only, there were 43 genes not found in the WBC samples. As might be expected, these genes were among those known to be specifically or highly expressed in erythrocytes. The top ones which came up by Fold Change (FC) analysis were the RBC proteins (erythrocyte membrane protein, hemoglobin zeta, glycophorin, selenium binding protein, and ALAS2).

Gene lists were generated first by filtering for genes whose expression resulted in a present call in all 3 samples of the set. Secondly, an analysis was performed to find genes that gave a present call in only one sample set (i.e. WBC only or 100 pm of the globin blockers only). These are the genes uniquely found in one preparation protocol only. Finally, a fold change analysis was performed examining the expression of genes expressed in common to both sample sets. A measurement of the differences in gene expression values between the two groups was generated, where the differences are significant at a p value of less than 0.001 as measured by a Two-Tailed T test.

In an analysis of the FC differentials between WBC and whole blood RNA+Blockers, the genes that were very highly up-regulated in whole blood RNA+Blockers tended to be related to RBC but some were unknown with regard to cell type. Of particular interest were the very high levels of alpha synuclein in the whole blood preparations. Genes which were very highly up-regulated in WBC compared to whole blood RNA+Blockers were almost uniformly ribosomal protein genes.

In short using the blockers is a vast improvement on the whole blood protocol alone and might also be implemented by using blockers to other highly expressed RBC proteins, including delta-aminolevulinate synthetase 2 (ALAS2), Selenium Binding Protein, Glycophorin and some of the other hemoglobins.

### Example 4

### Design and Evaluation of Oligonucleotide Blockers for Use with Primate Whole Blood Samples

Human α-globin blocker oligomers and primate β-globin blocker oligomers were tested for the ability to bind to α-globin and β-globin mRNA and block primer-directed reverse transcriptase of globin mRNAs in Cynomologus monkey whole blood preparations.

The nucleotide sequences encoding human α-globin and β-globin were evaluated for consensus to the Rhesus monkey and the Cynomolgus monkey α- and β-globin nucleotide sequences, respectively. The primate α-globin nucleotide sequences matched the human α-globin nucleotide sequence. For this reason, previously evaluated human α-globin blocking oligomers 04 and 05 were used, but 04 was lengthened as follows: UUUGCCGCCCACUCAGACUUUAU (SEQ ID No. 34, which is the same as SEQ ID No. 4, plus three additional nucleotides at the 3′ end). The comparison of the primate β-globin nucleotide sequence to the human β-globin nucleotide sequence revealed a one base pair difference. Three 2′-O-methyl primate β-globin blocking oligomers were designed and tested for their ability to effectively block reverse-transcription of primate β-globin mRNA. Evaluations were based on the results of Q-PCR and cRNA data. The β-globin blocking oligomers designed and tested are listed below.

2′-O-methyl β-globin blocking oligos
CyB1 (SEQ ID No. 35)
5′-mGmGmCmAmGmAmAmUmCmCmAmGmAmUmCmCmUmCm
AmAmGmGmG-3′
CyB2 (SEQ ID No. 36)
5′-mCmAmUmAmAmUmAmUmCmCmCmCmCmAmGmUmUmCm
AmGmUmG-3′
CyB3 (SEQ ID No. 37)
5′-mGmGmAmCmAmGmCmAmAmGmAmAmAmGmUmGmAmGmCmUmUm
UmG-3′

To perform the analysis, Cynomologus monkey blood is collected in EDTA tubes (1 tube of 10 ml blood/primate). Whole blood is then aliquoted in PAXgene™ blood tubes and tubes are processed according to the PAXgene™ Blood RNA Kit Handbook to obtain total RNA. The PAXgene™ T Blood RNA Kit stabilizes nucleic acids in blood including α- and β-globin RNA.

Following RNA extraction, reverse transcription is performed using 5 μg of total RNA per reaction. The reverse transcription step is performed according to the Affymetrix protocol with the exception that 2′-O-methyl modified globin-blocking oligomers were added to the reactions at the primer-annealing step. Table 3 provides sample descriptions. Sample CyP1 was used as a control.

TABLE 3

| | 2′-O-methyl α- | | 2′-O-methyl β- | |
|---|---|---|---|---|
| | globin blocking | | globin blocking | pmol/ |
| Sample | oligos | pmol/reaction | oligos | reaction |
| CyP1 | None | N/A | None | N/A |
| (control) | | | | |
| CyP2 | SEQ ID 34, | 90 | CyB1 (SEQ | 100 |
| | SEQ ID 5 | | ID No. 35) | |
| CyP3 | SEQ ID 34, | 90 | CyB2 (SEQ | 100 |
| | SEQ ID 5 | | ID No. 36) | |
| CyP4 | SEQ ID 34, | 90 | CyB3 (SEQ | 100 |
| | SEQ ID 5 | | ID No. 37) | |

Aliquots of approximately 0.9 μg of cDNA/sample were used for Q-PCR. Q-PCR was used to assess the ability of the 2′-O-methyl α-globin oligomers and 2′-O-methyl-β-globin oligomers to block reverse transcription of α-globin and α-globin mRNA. The Q-PCR data was analyzed by comparing the average C_{T} of each test sample (CyP2, CyP3, and CyP4) to that of the unblocked whole blood cDNA control (CyP1). An increase in the average C_{T} value for each blocked sample compared to the average C_{T} value for the unblocked sample indicated that the blockers were successful in blocking α-globin mRNA or β-globin mRNA reverse transcription compared to the control samples. All test samples had a higher average C_{T} value than the corresponding control average C_{T} value indicating that all oligomers blocked the reverse transcription of globin RNA in the test samples.

TABLE 4

| Sample | | Monkey α-globin |
|---|---|---|
| Name | 2-O-methyl oligos | Average C_{T} |
| CyP1 (control) | N/A | 17.32 |
| CyP2 | SEQ ID 34, SEQ ID 5, & SEQ ID | 18.06 |
| | No. 35 | |
| CyP3 | SEQ ID 34, SEQ ID 5, & SEQ ID | 18.99 |
| | No. 36 | |
| CyP4 | SEQ ID 34, SEQ ID 5, & SEQ ID | 18.44 |
| | No. 37 | |
| Sample | | Monkey β-globin |
| Name | 2-O-methyl Oligos | Average C_{T} |
| CyP1 (control) | N/A | 20.16 |
| CyP2 | SEQ ID 34, SEQ ID 5, & SEQ ID | 27.99 |
| | No. 35 | |
| CyP3 | SEQ ID 34, SEQ ID 5, & SEQ ID | 27.35 |
| | No. 36 | |
| CyP4 | SEQ ID 34, SEQ ID 5, & SEQ ID | 33.20 |
| | No. 37 | |

cDNA is transcribed to cRNA using the Affymetrix standard in vitro transcription (IVT) protocol. The quantity of cRNA is assessed by an A260 measurement. The CyP1 control sample was expected to have the highest total yield because no transcripts were blocked in that sample. Any yield under 25 μg would have been considered poor. The total yields for all test samples were high with the CyP2 sample performing statistically as well as the control sample. Samples CyP3 and CyP4 yielded less total cRNA than the CyP2 sample, but those yields were still considered satisfactory. The cRNA quality and 2′-O-methyl oligomers ability to block α-globin and β-globin was assessed on a 1× MOPS, 1.25% agarose gel. The criteria for acceptance was the lack of a ≦0.6 Kb band which was shown in Example 1 to correspond to globin. The presence of a band 0.6 Kb or smaller would have suggested partial, rather than complete, blockage of globin transcription. The gel showed that globin was completely blocked in all test samples.

TABLE 5

| | Total Yield | |
|---|---|---|
| Sample Name | (μg) | Fold Increase |
| CyP1 (control) | 79.01 | 17.37 |
| CyP2 | 84.03 | 18.47 |
| CyP3 | 70.97 | 15.60 |
| CyP4 | 65.84 | 14.47 |

The Q-PCR and cRNA data indicated all oligomers bound and effectively blocked the reverse transcription of the α- and β-globin RNA. The CyB1 oligomer was designed to bind closest to the 3′ end of the β-globin mRNA. It blocked the transcription of β-globin RNA as effectively as the other β-globin blocking oligomers, and it resulted in the highest yield of cRNA. For this reason, CyB1 (SEQ ID No. 35) appears to be the most effective β-globin blocking oligomer evaluated.

### Example 5

### Gene Expression Analysis of Primate Whole Blood Samples With and Without Interfering Molecules

Gene expression analysis of Cynomologus monkey white blood cells, whole blood, and whole blood with blocking oligomers is performed on the Affymetrix HG_U133A GeneChip® array. RNA is obtained from white blood cells by lysing the erythrocytes in whole blood and extracting total RNA using the Qiagen RNeasy Kit according to the manufacturer's instructions. The PAXgene Blood RNA kit is used to extract total extracted from whole blood preparations and is used according to manufacturer's instructions. Total RNA samples are processed for use with Affymetrix HG_U133A GeneChip® arrays according to Affymetrix standard protocols with the exception that blocking oligomers are added to sample PAX_{—}100 during the primer-annealing step of reverse transcription of RNA to cDNA. Table 6 provides sample information.

TABLE 6

| | | α-globin | | β-globin | |
|---|---|---|---|---|---|
| Sample | RNA | blocking | | blocking | |
| Name | treatment | oligomers | Concentration | oligomers | Concentration |
| WBC | White Blood | 0 | N/A | 0 | N/A |
| | Cell | | | | |
| | Preparation | | | | |
| | Rneasy | | | | |
| PAX_0 | PAXgene | 0 | N/A | 0 | N/A |
| PAX_100 | PAXgene | SEQ ID 34, | 90 pmol/ | CyB1 (2′-O- | 100 pmol/ |
| | | SEQ ID 5 (2′- | reaction | methyl | reaction |
| | | O-methyl | | modified) | |
| | | modified) | | (SEQ ID No. | |
| | | | | 35) | |

Each sample is run on three Affymetrix HG_U133A GeneChip® arrays according to Affymetrix standard protocols. The samples showed lower present calls than those described in Example 2 because of cross-species hybridization. The white blood cells sample showed approximately 21.5% present calls (˜9,000 out of ˜22,000 genes or ˜24%). The whole blood sample without blocking oligomers showed approximately 17.9% present calls. In comparison, the whole blood sample with blocking oligomers showed approximately 26.0% present calls. This data showed that more genes were detected in the whole blood with blocking oligomers sample than the white blood cell sample or the whole blood without blocking oligomers sample.

Correlation color maps and PCA graphs (not shown) also demonstrated that there was a high level of concordance between the present call genes of the white blood cell sample and whole blood with blocking oligomers sample.

### Example 6

### Evaluation of Oligonucleotide Blockers for Use with Canine Whole Blood Samples

Canine blocking oligomers were designed and evaluated for their ability to effectively block reverse transcription of globin mRNA. Canine blood is collected and processed with the PAXgene™ Blood RNA Kit according to the manufacturer's instructions. mRNA is reverse transcribed and 2′O-methyl modified blocking oligomers are added at 100 pmol/reaction during the primer-annealing step for the test samples. The α- and β-globin blocking oligomers designed and tested are listed below. Sample descriptions are listed in Table 7.

Canine Blocking Oligomers

CANaG.04 (SEQ ID No. 38)
5′-mGmCmAmGmGmCmAmGmCmCmCmAmCmUmCmAmGmAmCmUmUmUm
AmUmUmC-3′
CANaG.05 (SEQ ID No. 39)
5′-mUmCmAmAmAmCmAmUmCmAmGmGmAmAmGmUmGmCmAmGmGmGmCm
AmCmC-3′
CANaG.06 (SEQ ID No. 40)
5′-mGmCmGmCmAmGmGmAmAmGmCmGmGmCmCmCmAmGmGmGmCmAmGm
G-3′
CANbG.01 (SEQ ID No. 41)
5′-mGmAmAmGmCmCmAmUmAmCmCmCmUmUmGmAmUmGmGmUmAmGmAm
C-3′
CANbG.02 (SEQ ID No. 42)
5′-mCmUmUmCmCmAmGmUmGmGmUmCmAmCmCmAmGmGmAmAmAmCmAm
G-3′
CANbG.03 (SEQ ID No. 43)
5′-mGmCmCmAmCmAmCmCmAmGmCmCmAmCmCmAmCmCmUmUmCmUm
G-3′

TABLE 7

| Sample | | |
|---|---|---|
| Name | α-globin blocking oligomers | β-globin blocking oligomers |
| K9P1 | 0 | 0 |
| K9P17 | CANaG4 | CANbG2, CANbG3 |
| K9P18 | CANaG4, CANaG5 | CANbG2, CANbG3 |
| K9P19 | CANaG4, CANaG5, CANaG6 | CANbG2, CANbG3 |
| K0P20 | CANaG4, CANaG5, CANaG6 | CANbG1, CANbG2, |
| | | CANbG3 |

Samples are assessed using Q-PCR as described in Example 2. The control sample (whole blood, no blockers) had the lowest average α-globin C_{T} and β-globin C_{T} values across all samples. This indicated that all blockers blocked the reverse transcription of globin RNA in the test samples compared to the control sample.
cDNA samples are transcribed to cRNA as described in Example 2. The whole blood sample without blocker oligomers had a total yield of 110.45 μg of cRNA. The blocked samples had lower yields, but all yields were above above 29 μg. Yields below 25 μg would have been considered as “failing”. For this reason, the total yields of all the test samples were considered satisfactory.
cRNA was also run on a 1×MOPS, 1.25% agarose gel to determine whether blocking oligomers had completely blocked reverse transcription of globin mRNA. A faint band of approximately 0.6 Kb in the lane of the gel corresponding to sample K9P 17 suggested that only partial blockage occurred for this sample. The lanes of the gel corresponding to the other test samples showed no bands which suggested that complete blockage of reverse transcription of globin mRNA transcripts occurred. For this reason, it appears that a combination of α-globin blocking oligomers blocks reverse transcription more effectively than α-globin blocking oligomer CANaG.04 blocks alone.

### Example 7

### Gene Expression Analysis of Canine Whole Blood Samples with and without Interfering Molecules

Gene expression analysis of canine white blood cells, whole blood, and whole blood with globin blockers is performed using the Affymetrix Canine GeneChip® array platform. The experiment parallels the experiment described in Example 5. The white blood cell samples are obtained by lysing erythrocytes, and total RNA is extracted using the Qiagen RNeasy Kit. Total RNA is extracted from whole blood samples using the PAXgene Blood RNA Kit. Sample descriptions are provided in Table 8.

TABLE 8

| | | | Blocking oligomer |
|---|---|---|---|
| Sample | | | concentration |
| set name | Starting sample | Blocking oligomers | (pmol/reaction) |
| WBC | White blood | None | N/A |
| | cells | | |
| PAX_0 | Whole blood | None | N/A |
| PAX_10 | Whole blood | CANaG4, CANaG5, | 10 |
| | | CANaG6; CANbG2, | |
| | | CANbG3 | |
| PAX_100 | Whole blood | CANaG4, CANaG5, | 100 |
| | | CANaG6; CANbG2, | |
| | | CANbG3 | |
| PAX_200 | Whole blood | CANaG4, CANaG5, | 1000 |
| | | CANaG6; CANbG2, | |
| | | CANbG3 | |

Samples are processed as described in Example 5, and samples are hybridized to one Canine GeneChip® array each. The WBC sample set had the highest percent present calls at 34.8%. The PAX_{—}0 sample set had the lowest percent present calls at 16.7%. The PAX_{—}10, PAX_{—}100, and PAX_{—}200 sample sets had percent present calls of 29.9%, 31.9%, and 29.3% respectively. The data shows that the percent present calls substantially increased in whole blood cell samples when globin blocking oligomers were added.

The PAX_{—}100 sample set showed the highest level of concordance with the WBC sample set compared to the other whole blood cell preparations. The concordance of present call genes between the WBC sample set and the PAX_{—}100 sample set was 86.3%. The concordance of present call genes between the WBC sample set and the PAX_{—}0 sample set was 46.5%. The data shows that gene expression data was most similar between the white blood cell sample and the whole blood with 100 pmol of blocking oligomers sample.

### Example 8

### Gene Expression Analysis of Rat Whole Blood Samples: A Comparison of PAXgene® and TRIzol® Protocols for Analysis of Differential Gene Expression with and without Globin Reduction

The objectives of this study were to evaluate the effectiveness of the globin reduction protocol on rat whole blood samples, and to evaluate the level of improvement in the measurement of gene expression differences in globin reduction protocol treated samples to that of untreated samples when compared to the WBC protocol.

Fifteen rats were treated with saline (3 ml/kg, ip) and fifteen animals were treated with LPS (10 mg/kg in a volume of 3 ml/kg, ip), and RNA was isolated from rat blood using (1) the University of North Carolina RBC lysis protocol (“UNC”) (Yang et al., 2002, Expression profile of leukocyte genes activated by anti-neutrophil cytoplasmic autoantibodies (ANCA), Kidney Intl., 62(5): 1638-49), whole blood mixed with TRIzol® protocol and the PAXgene® standard isolation protocol. mRNA is reverse transcribed and 2′O-methyl modified blocking oligomers are added at 400 pmol/reaction of 5 μg total RNA during the primer-annealing step for the test samples. The blockers used were as follows:

Rat alpha globin 10 (SEQ ID No. 45)
5′-mGmGmAmCmGmGmAmAmGmAmAmGmGmGmCmCmUmGmGmUmCmAm
G-3′
Rat alpha globin 11 (SEQ ID No. 46)
5′-mGmCmAmAmGmCmCmCmGrnAmCmAmGmGmAmGmGmUmGmGmCm
U-3
Rat beta globin 01 (SEQ ID No. 10)
mGmCmAmGmUmGmAmAmAmGmUmAmAmAmUmGmCmCmUmU
Rat beta globin 02 (SEQ ID No. 11)
mGmAmCmAmAmCmAmAmCmUmGmAmCmAmGmAmUmGmCmUmCmUmC
Rat beta globin 04 (SEQ ID No. 13)
mGmCmUmCmUmCmUmUmGmGmGmAmAmCmAmAmUmUmGmAmCmC
Rat beta globin 05 (SEQ ID No. 14)
mGmGmCmAmCmUmGmGmCmCmAmCmUmCmCmAmGmCmCmAmCmC

Each of the samples is then hybridized for 24 hours at 45° C. to one RGU34A GeneChip® array, and the arrays were analyzed using Microarray Suite 5.0 software (Affymetrix) using the following settings: scaling=all probe sets @ TGT 100, Normalization factor=1, alpha 1=0.04, alpha 2=0.06, tau=0.015, gamma 1L=0.0025, gamma 1H=0.0025, gamma 2L=0.003, gamma 2H=0.003, and perturbation=1.1.

A list of probe sets was obtained from the ASCENTA™ system (Gene Logic, Inc., Gaithersburg, Md.) which were considered members of either the cytokine gene family (53 members) or the GPCR gene family (277 members). A comparison of the log₂ transformed Geomean data showed a significantly increased correlation (R2) between TRIzol® or PAXgene® samples treated with the globin reduction protocol and the UNC protocol than with untreated whole blood samples. This indicates a significant improvement in the accuracy of gene expression measurements occurs when globin message is removed.

A subset of genes which are members of eight inflammatory pathways (tumor necrosis factor (TNF), cytokine inflammatory response, interleukin-6 (IL-6), cytokine network, inflammatory response, cytotoxic T lymphocytes (CTL) immune response, transforming growth factor beta (TGF beta) and mitogen-activated protein kinases (MAPK)), and which showed significant gene expression differences between control and LPS trated WBC samples was examined. The magnitiude of measured gene expression differences between control and LPS-treated samples observed in the WBC sample set was compared to that of the TRIzol® and PAXgene® sample sets. It was observed that for both the TRIzol® and PAXgene® sample sets treated with the globin reduction protocol, there was an increase in the correlation of the calculated magnitude of gene expression differences measured between the control and LPS treated samples with that of the WBC sample set. That is, the correlation of the magnitude of fold changes is much higher and the direction of the fold change in expression is more consistent to that of the WBC sample after globin reduction (for both the TRIzol® and PAXgene® sample sets).

In summary, treatment of the TRIzol® and PAXgene® RNA samples with the globin reduction protocol provides significant benefits to the accurate measurement of differential gene expression in WBCs. By removing the majority of alpha and beta-globin cRNA from the array hybridization solution, the sensitivity of gene detection and the accuracy and reproducibility of measured gene expression increases substantially. Since the globin reduction protocol involves protocol steps in addition to TRIzol® and PAXgene® RNA isolation, the protocol also has the advantage of no WBC isolation.

### Example 9

### Blocking of β-Globin, β-Globin and γ-Globin During Reverse Transcription to Enhance Whole Blood Gene Expression Profiling in Human Samples

The objectives of this study were (1) to measure the effectiveness of the globin reduction protocol on human whole blood samples which have a large range of reticulocyte counts ranging from 0.2% to 2.5%; (2) to determine if the blocking method remains effective in samples containing either very low or very high amounts of globin mRNAs; and (3) to compare the globin reduction protocol of the present invention using 2′O-methyl chemistry modified oligomers as gene specific blockers to Affymetrix's recently published RNase H-based globin reduction protocol (Affymetrix Technical Note An Analysis of Blood Processing Methods to Prepare Samples for GeneChip® Expression Profiling (2003)).

Blood was collected from each of 6 different donors and processed for a complete blood count analysis including a reticulocyte count analysis. Total RNA was isolated from part of each sample utilizing each of (1) the “RNeasy Midi Protocol for Isolation of Total Cellular RNA from Whole Blood” (Qiagen) protocol, termed “WBC” in following paragraphs (including the optional on-column “RNase-free DNase Set” (Qiagen) DNase I treatment digestion); (2) the TRIzol® RNA isolation protocol (Invitrogen); and (3) the “PAXgene™ Blood RNA Kit” (PreAnalytiX) protocol (including the optional on-column “RNase-free DNase Set” (Qiagen) DNase I treatment digestion).

Each of the 6 WBC total RNA, 6 TRizol® RNA and multiple PAXgene™ total RNA samples were individually assessed for RNA quality on the Agilent 2100 Bioanalyzer system using the “RNA 6000 Nano LabChip Kit” (Agilent), and then concentrated to a concentration of greater than 1 μg/μl using either the “RNeasy Mini Kit” protocol (Qiagen) or the “RNeasy MinElute Cleanup Kit” protocol (Qiagen). Concentrated RNA samples were prepared following the standard protocol for sample preparation for GeneChip® analysis as listed in the “GeneChip® Expression Analysis Technical Manual—Chapter 2: Eukaryotic Sample and Array Processing” manual (Affymetrix). Additionally, aliquots of each TRIzol® and PAXgene™ total RNA samples were treated with either the globin reduction protocol of the present invention or the Affymetrix globin reduction protocol as follows:

Globin Reduction Protocol method of the present invention: At the start of the first strand cDNA synthesis reaction, 5 μg aliquots of both PAXgene™ and TRIzol® total RNA were annealed simultaneously to 100 pmol of the T7-oligo (dT) primer and 5 μl of a modified oligonucleotide (oligo) mix containing 90 pmol each of the following 5 different globin mRNA blocking oligonucleotides (two alpha blockers, two beta blockers and 1 gamma blocker, each at 90 pmol per reaction):

Human beta globin 02 (SEQ ID No. 2)
5′-mGmGmAmCmAmGmCmAmAmGmAmAmAmGmCmGmAmGmCmUmUmUmG-
3′
Human alpha globin 05 (SEQ ID No. 5)
5′-mCmCmAmCmCmGmAmGmGmCmUmCmCmAmGmCmUmUmAmAmCmGmG-
3′
Human alpha globin 04 (extended) (SEQ ID No. 34)
5′-mUmUmUmGmCmCmGmCmCmCmAmCmUmCmAmGmAmCmUmUmUmAmU-
3′
Human beta globin 01 (extended) (SEQ ID No. 44)
5′-mGmGmCmAmGmAmAmUmCmCmAmGmAmUmGmCmUmCmAmAmGmGmC-
3′
Human gamma globin 07 (SEQ ID No. 7)
5′-mUmGmUmGmAmUmCmUmCmUmCmAmGmCmAmGmAmAmUmAmGmAmU-
3′

Each annealing reaction was done in a total volume of 12 μl at 70° C. for 10 minutes (for a total of 2 sets of 6 samples). All 12 “treated” samples were then prepared for GeneChip® analysis following the remainder of the protocol as listed in the “GeneChip® Expression Analysis Technical Manual—Chapter 2: Eukaryotic Sample and Array Processing” manual (Affymetrix).

Affymetrix's Globin Reduction Protocol method: Prior to the start of the first strand cDNA synthesis reaction, 5 μg aliquots of both PAXgene™ and TRIzol® total RNA were annealed simultaneously to 15 pmoles each of 2 different alpha globin 3′ end antisense primers and 40 pmoles of a beta globin 3′ antisense primer. Each annealing reaction was done in a total volume of 10 μl at 70° C. for 5 minutes (for a total of 2 sets of 6 samples). Each annealed sample was then digested with 2 Units RNase H (Invitrogen) in a total reaction volume of 20 μl at 37° C. for 10 minutes. The RNase H-digested total RNA samples were then cleaned and concentrated in a volume of 11 μl using the IVT cRNA Cleanup Spin Column from the GeneChip® Sample Cleanup Module (Affymetrix). All 12 “treated” samples were then prepared for GeneChip® analysis following the remainder of the protocol as listed in the “GeneChip® Expression Analysis Technical Manual—Chapter 2: Eukaryotic Sample and Array Processing” manual (Affymetrix).

Each WBC, TRIzol®, and PAXgene™ RNA sample (including samples treated with either the Globin Reduction Method of the Invention or the Affymetrix Globin Reduction protocol) was then hybridized for 16 hours at 45° C. to one Hu133A array each. Each array was washed, stained, and scanned (on a single scanner) according to the “GeneChip® Expression Analysis Technical Manual—Chapter 2: Eukaryotic Sample and Array Processing” manual (Affymetrix) (see SOPs 3037v2 and 3008v3). Each array image was assessed for quality using Gene Logic's proprietary QC workbench program and then analyzed using Microarray Suite software (Affymetrix). The MAS 5.0 analysis settings used were as follows: scaling=all probe sets @ TGT 100, Normalization factor=1, alpha 1=0.05, alpha 2=0.065, tau=0.015, gamma 1L=0.0045, gamma 1H=0.0045, gamma 2L=0.006, gamma 2H=0.006, and perturbation=1.1.

A typical range for the length of the cRNA targets, between 200 and 4,000 bases, can be seen for the WBC preparation. With the preparation from the PAXgene™ system and TRIZOL®, a dominant, ˜600 bp band is apparent and the relative intensity in the cRNA distribution is lower than that observed with WBC preparations. The dominant ˜600 bp band is significantly reduced and is not apparent in the images generated from samples prepared with either of the globin reduction approaches. In addition, the length of the cRNA target distribution in PAXgene™ or TRIzol® samples treated with the globin reduction protocol of the invention is again compatible to the WBC cRNA target. However, there appears to be a slight reduction in the length of cRNA target distribution in PAXgene™ or TRIzol® samples treated with the Affymetrix's RNase H based globin reduction protocol.

The highest expressed genes in the PAXgene™ preparations compared to those expressed in erythrocyte lysed preparations are the globin transcripts (data not shown). The dominant ˜600 bp band is attributed to amplification of globin mRNAs from reticulocytes that are present in the whole blood preparations but removed in other methods. To target the globin transcripts, the globin reduction protocol of the invention utilizes five different gene specific blocking oligomers for globin transcripts α-, β-, and γ-globin that were designed against HBA1, HBA2, HBB, and HBG1 respectively). The Affymetrix globin reduction protocol utilizes primers which target α- and β-globin transcripts only (specific for the HBA1, HBA2, and HBB sequences). However, each blocking approach tested, removed the predominant ˜600 bp band completely. It is worth noting that the ˜600 bp band is not detectable in the total RNA preparations, it only appeared after the cRNA amplification process was performed. The relative reduction in cRNA intensity and the apparent length of the TRIzol® and PAXgene™ samples in gel images may result from the competition between the abundant globin messages and the remaining transcripts during amplification and labeling or may simply be a result of dilution of non-globin cRNAs in the sample by a large amount of globin cRNA.

Consistently lower “Percent Present calls” and higher MM>PM probe-pair counts were observed in the TRIzol® and PAXgene™ samples (data not shown). Since the reduction of the ˜600 bp band is correlated with increased “Percent Present calls” and lower MM>PM probe-pair counts, the reduced sensitivity in the PAXgene™ and TRizol® experiments is most likely due to the presence of the dominant band in the amplified cRNA target present in the whole blood RNA preparations.

In order to determine the efficiency of globin transcript depletion prior to or during cDNA synthesis, the α-, γ-, and γ-globin gene expression values for each sample were extracted. In some cases up to 6 different probe sets were used to measure the average gene expression of a single globin gene. Samples with higher levels of reticulocytes displayed larger globin signal values, however, both globin reduction methods decreased the measured gene expression signal values for their expected transcripts. The blocker cocktail for the method of the invention reduced the signal value of the α, β, and γ-globin probe sets to approximately the same signal value range or below the range of values measured in the WBC preparations. The Affymetrix globin reduction protocol, however, did not reduce the signal values of the globin probe sets as significantly as the instant globin reduction protocol.

It is worth noting that the Gene Logic globin reduction protocol actually reduced the expression signal of β-globin to a level slightly below the values observed with the WBC protocol. Also, as expected, the Affymetrix globin reduction protocol had little effect on the γ-globin values (since it does not specifically target the γ-globin transcripts for RNase H digestion). Both globin reduction protocols showed a similar effectiveness at reducing globin signal across samples from donors displaying a wide range of reticulocyte counts. This indicates that both protocols should be effective in reducing globin for a variable donor sample population.

A Student's t-test was used to identify genes that showed differential expression between WBC total RNA as the baseline expression and the different whole blood total RNA and globin depletion approaches. The majority of probe sets in each comparison displayed smaller than two-fold change differences. However, the comparisons of TRIzol® and PAXgene™ total RNA with the WBC preparation, revealed 843 and 1020 probe sets respectively with a 2-fold expression difference in either direction at a p-value of <0.01. The number of significant expression differences was reduced with the globin reduction approach of the present invention from 843 to 124 probe sets in TRIzol® samples and 1020 to 391 probe sets in PAXgene™ samples respectively. The number of significant expression differences was also reduced, but not as significantly, by the Affymetrix globin reduction protocol approach from 843 to 726 probe sets in TRIzol® samples and 1020 to 799 probe sets in PAXgene™ samples respectively. We observed a large increase in the number of significant negative fold changes for samples treated with this method indicating non-specific or off-target effects are occurring during the Affymetrix protocol. Clearly, in this analysis the instant globin reduction approach is the best performing protocol and the data suggests, if any, a low number of off-target effects in samples treated with this protocol.

Two sets of cell type specific and gene family specific signature genes were used to correlate the gene expression data produced by the different protocols tested based on blood specific genes. The red blood cell specific genes were more highly expressed in any of the whole blood total RNA protocols. The correlation of granulocyte and mononuclear cell specific transcripts for WBC vs. untreated TRIzol® samples is R²=0.90 and for WBC vs. untreated PAXgene™ samples is R²=0.83, but this correlation was increased in any of the globin reduction protocol treated samples of the invention to greater than 0.99. The highest correlation of R²=0.99 was observed in the TRIzol® samples treated with the globin reduction protocol of the present invention.

Interestingly, TRIzol® and PAXgene™ samples treated with Affymetrix's RNase H based globin reduction protocol performed very poorly in this particular analysis. The correlation of granulocyte and mononuclear cell specific gene expression values for WBC vs. TRIzol®⁺RNase H is R²=0.66 and WBC vs. PAXgene™+RNase H is R²=0.63. For this gene set it is clear that off-target effects have actually reduced the correlation to WBC sample data.

Similar results were observed for the second set of signature genes. The correlation of gene expression values for WBC vs. untreated TRIzol® or PAXgene™ samples is R²=0.85 and 0.83 respectively. This correlation was increased to 0.97 and 0.94 for TRIzol® and PAXgene™ samples respectively treated with the globin reduction protocol of the present invention. Additionally, in contrast to the granulocyte and mononuclear cell specific genes, there was an increase in the correlation observed in TRIzol® and PAXgene™ samples treated with Affymetrix's protocol compared to WBC (R²=0.90 and 0.89 respectively).

To further determine the number of probe sets displaying possible protocol off-target effects, the ratio of the geometric means (for each probe set) for each globin reduction protocol (i.e. TRIzol®+blockers, TRIzol®+RNase H, etc) was compared to the untreated PAXgene™ or TRIzol® sample data and a Student's t-Test was performed for each comparison to determine the significance of any measured expression differences. Finally, a filtered list of probe sets was determined that included only those probe sets that had a higher geometric mean signal value in the WBC sample set than in the untreated PAXgene™ or TRIzol® sample sets. Probe sets that showed a significant decrease in treated vs. untreated samples (a signal decrease of more than 1.5 fold, p<0.05) and that were measured as expressed at a higher level in WBC samples than the untreated samples were counted. Only 6 and 2 out of ˜22000 probe sets met these criteria for PAXgene™ and TRIzol® samples treated with the globin reduction protocol of the present invention. However, 329 and 520 probe sets met these same criteria for PAXgene™ and TRIzol® samples treated with Affymetrix's globin reduction protocol. The conclusion from this analysis is that Affymetrix's RNase H based protocol causes a significant and large number of off-target effects. This could be due to the nature of the protocol itself: by employing an enzymatic reaction in samples which could contain fragments of genomic DNA there is the potential for many different non-globin mRNA digestions to occur.

In summary, GeneChip® array data obtained from the 6 different whole blood samples prepared with either the instant or Affymetrix's globin reduction protocol was compared to data from unblocked whole blood total RNA samples. The performance of these different protocols was evaluated on numerous parameters prior to and after hybridization on GeneChip® arrays. Expression data analysis revealed that instant protocol performed better than Affymetrix's protocol in all analyses except % Present and concordance analyses of PAXgene™ samples. In addition, the instant protocol significantly increased the sensitivity and reproducibility of whole blood sample microarray data, was the easiest protocol to implement in production, and is the most amenable to automation.

Using the transcription blocking approach of the present invention on samples processed for the Affymetrix GeneChip® platform, the number of measurable genes, as related to those derived from total RNA from whole blood preparations, increased from 66% to 86%. A comparison of genes that were gained using the transcription blocking protocol to genes that were measured as Present calls in the same sample processed using a reticulocyte lysis protocol, resulted in a 96% overlap between the two protocols. This suggests that the biological integrity of gene expression is maintained and that the gene expression analysis of the more relevant peripheral white blood cells can be obtained. The average coefficient of variation was reduced in the transcription blocked protocol by 3.9% without any significant changes in signal to noise ratios or 5′-3′ ratios for the GAPDH or β-actin reference genes. One concern when using the blocking approach is that there may be “off target” silencing of non-blocker targeted transcription. However, analysis of the resultant data showed that of the ˜22,000 genes tiled on the microarray, a maximum of 6 demonstrated possible off-target effects. These results demonstrate that the use of whole blood total RNA, stabilized at the time of collection, can be efficiently used as a sample for whole genome gene expression profiling without loss of sensitivity and reproducibility.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of improving gene expression analysis of blood or tissue sample having a high erythrocyte content comprising the steps of:
(a) obtaining a sample of RNA from said blood or tissue;
(b) adding one or more red blood cell (RBC) nucleic acid sequence-specific interfering molecules to said sample;
(c) amplifying the RNA transcripts in said sample; and
(d) determining the gene expression profile of said sample.

2. The method of claim 1, wherein said one or more RBC nucleic acid sequence-specific interfering molecules block reverse transcription of one or more RBC mRNA transcript species into cDNA.

3. The method of claim 2, wherein said blood or tissue is whole blood.

4. The method of claim 3, wherein said one or more RBC mRNA transcript species is globin and said one or more RBC nucleic acid sequence-specific interfering molecules is a globin nucleic acid sequence-specific interfering molecule.

5. A method of inhibiting amplification during a nucleic acid amplification process of one or more RBC RNA transcript species in a sample containing RNA, comprising the steps of:
(a) adding one or more RBC nucleic acid sequence-specific interfering molecules to said sample; and
(b) amplifying the RNA transcripts in said sample.

6. The method of claim 5, wherein said one or more RBC nucleic acid sequence-specific interfering molecules block reverse transcription of one or more RBC mRNA transcript species into cDNA.

7. The method of claim 6, wherein said sample is obtained from whole blood.

8. The method of claim 7, wherein said one or more RBC mRNA transcript species is globin and said one or more RBC nucleic acid sequence-specific interfering molecules is a globin nucleic acid sequence-specific interfering molecule.

9. A method of inhibiting amplification of one or more red blood cell RNA transcript species in a sample that impede gene expression analysis of other transcript species in the sample, comprising:
(a) adding one or more red blood cell nucleic acid sequence-specific interfering molecules to the sample; and
(b) amplifying transcripts in the sample in the presence of said one or more red blood cell nucleic acid sequence-specific interfering molecules.

10. The method of claim 9, wherein said amplification comprises reverse transcription of said transcript species.

11. The method of claim 9, wherein said sample is whole blood, or a RNA preparation obtained from a tissue having a high erythrocyte content, wherein said tissue is optionally selected from the group consisting of spleen, bone marrow, placenta, vascularized tumor, angioid tumor, adipose, lung, muscle, pancreas, heart, liver and hemorrhagic tissues.

12. The method of claim 9, wherein said gene expression analysis is a quantitative.

13. The method of claim 9, wherein said red blood cell transcript species are selected from the group consisting of transcripts for ribosomal proteins L3 (RPL3L), L6 (RPL6), L7 (RPL7), L7a (RPL7A), L9 (RPL9), L10a (RPL10A), L11 (RPL11), L12 (RPL12), L13a) RPL13A), L17 (RPL17), L18 (RPL18), L19 (RPL19), L21, L23a (RPL23A), L24 (RPL24), L27 (RPL27), L27a (RPL27A), L28 (RPL28), L30 (RPL30), L31 (RPL31), L32 (RPL32), L34 (RPL34), L35 (RPL35), L37 (RPL37), L37a (RPL37A), L41 (RPL41), S2 (RPS2), S3a (RPS3A), S5 (RPS5), S6 (RPS6), S7 (RPS7), S10 (RPS10), S11 (RPS11), S13 (RPS13), S16 (RPS16), S17 (RPS17), S18 (RPS18), S23 (RPS23), S24 (RPS24), S27a (RPS27A), S31 (RPS31), SM, large ribosomal protein PO(RPLPO), flavin reductase (BLVRB), ferrochelatase (FECH), myosin light protein (MYL4), synucleic alpha (SNCA), delta-aminolevulinate synthetase 2 (ALSA2), selenium binding protein 1 (SELENBP1), erythrocyte membrane protein bands 4.2 (EPB42) and 4.9 (EPB49), glycophorin C (GYPC), antioxidant protein 2 (AOP2), beta actin (ACTB), gamma actin 1 (ACTG1), vimentin (VIM), adipocyte fatty acid binding protein 4 (FABP4), eukaryotic translation elongation factor 1 alpha 1 (EEF1E1), translationally-controlled 1 tumor protein (TPT1), ubiquitin C (UBC), ferritin light polypeptide (FTL), leukocyte receptor cluster (LRC) member 7 (LENG7), beta-2-microglobulin (B2M), glyceraldehyde-3-phosphate dehydrogenase (GAPD), replication factor C (activator 1) (RFC1), heterogeneous nuclear ribonucleoprotein A1 (HNRPR1), Finkel-Bis kis-Reilly murine sarcoma virus (FBR-MuSV) ubiquitously expressed (fox derived) (FAU), ras homolog gene family member A (ARHA), cofilin 1 (non-muscle) (CFL1), ornithine (MGST1), early growth response 1 (EGR1), microsomal glutathione S-transferase 1 (MGST1), peptidylprolyl isomerase A (cyclophilin A) (PPIA), carcinoembryonic antigen-related cell adhesion molecule 5 (CEACAM5), galactoside-binding lectin 4 (LGALS4), liver fatty acid binding protein 1 ((FABP1), coatomer protein complex subunit gamma (immunoglobulin lambda joining 3) (COPG, IGLJ3), major histocompatibility complex class 1B (HLA-B), major histocompatibility complex class 1C (HLA-C), immunoglobulin heavy mu constant (IGHM), immunoglobulin kappa constant (IGKC), solute carrier family 25 member 3 (SLC25A3), H3 histone family 3A (H₃FA), normal mucosa of esophagus specific 1 (NMES1), heat shock 70 kDa protein 8 (HSPA8), hypothetical protein MGC14697 (MGC14697), polymeric immunoglobulin receptor (PIGR), and FK 506 binding protein 8 (FKBP8), hypothetical protein BC012775 (LOC91300), cold shock domain protein A (CSDA), F-box only protein 7 (FBX07), CGI-45 protein (CGI-45), makorin ring finger protein 1 (MKRN1), small EDRK-rich factor 2 (SERF2), pinin (PNN), SET domain bifurcated 1 anti-oxidant protein 2 (AOP2, SETDB1), nuclease sensitive element binding protein (NSEP1), glutathione peroxidase 1 (GPX1), MAX interacting protein 1 (MXI1), and ubiquitin B (UBB).

14. A method of enhancing quantitative gene expression analysis comprising inhibiting reverse transcription of one or more red blood cell transcript species in a sample that impede gene expression analysis of other transcript species in the sample, wherein said inhibiting comprises:
(a) adding one or more red blood cell nucleic acid sequence-specific interfering molecules to the sample; and
(b) reverse transcribing RNA in the sample in the presence of said one or more red blood cell nucleic acid sequence-specific interfering molecules.

15. The method of claim 14, wherein said sample is whole blood, or a RNA preparation obtained from a tissue having a high erythrocyte content, wherein said tissue is optionally selected from the group consisting of spleen, bone marrow, placenta, vascularized tumor, angioid tumor, adipose, lung, muscle, pancreas, liver, heart and hemorrhagic tissues.

16. The method of claim 14, wherein said red blood cell transcript species are selected from the group consisting of transcripts for ribosomal proteins L3 (RPL3L), L6 (RPL6), L7 (RPL7), L7a (RPL7A), L9 (RPL9), L10a (RPL10A), L11 (RPL11), L12 (RPL12), L13a) RPL13A), L17 (RPL17), L18 (RPL18), L19 (RPL19), L21, L23a (RPL23A), L24 (RPL24), L27 (RPL27), L27a (RPL27A), L28 (RPL28), L30 (RPL30), L31 (RPL31), L32 (RPL32), L34 (RPL34), L35 (RPL35), L37 (RPL37), L37a (RPL37A), L41 (RPL41), S2 (RPS2), S3a (RPS3A), S5 (RPS5), S6 (RPS6), S7 (RPS7), S10 (RPS10), S11 (RPS11), S13 (RPS13), S16 (RPS16), S17 (RPS17), S18 (RPS18), S23 (RPS23), S24 (RPS24), S27a (RPS27A), S31 (RPS31), SM, large ribosomal protein PO(RPLPO), flavin reductase (BLVRB), ferrochelatase (FECH), myosin light protein (MYL4), synucleic alpha (SNCA), delta-aminolevulinate synthetase 2 (ALSA2), selenium binding protein 1 (SELENBP1), erythrocyte membrane protein bands 4.2 (EPB42) and 4.9 (EPB49), glycophorin C (GYPC), antioxidant protein 2 (AOP2), beta actin (ACTB), gamma actin 1 (ACTG1), vimentin (VIM), adipocyte fatty acid binding protein 4 (FABP4), eukaryotic translation elongation factor 1 alpha 1 (EEF1E1), translationally-controlled 1 tumor protein (TPT1), ubiquitin C (UBC), ferritin light polypeptide (FTL), leukocyte receptor cluster (LRC) member 7 (LENG7), beta-2-microglobulin (B2M), glyceraldehyde-3-phosphate dehydrogenase (GAPD), replication factor C (activator 1) (RFC1), heterogeneous nuclear ribonucleoprotein A1 (HNRPR1), Finkel-Bis kis-Reilly murine sarcoma virus (FBR-MuSV) ubiquitously expressed (fox derived) (FAU), ras homolog gene family member A (ARHA), cofilin 1 (non-muscle) (CFL1), ornithine decarboxylase antizyme 1 (OAZ1), microsomal glutathione S-transferase 1 (MGST1), early growth response 1 (EGR1), microsomal glutathione S-transferase 1 (MGST1), peptidylprolyl isomerase A (cyclophilin A) (PPIA), carcinoembryonic antigen-related cell adhesion molecule 5 (CEACAM5), galactoside-binding lectin 4 (LGALS4), liver fatty acid binding protein 1 ((FABP1), coatomer protein complex subunit gamma (immunoglobulin lambda joining 3) (COPG, IGLJ3), major histocompatibility complex class 1B (HLA-B), major histocompatibility complex class 1C (HLA-C), immunoglobulin heavy mu constant (IGHM), immunoglobulin kappa constant (IGKC), solute carrier family 25 member 3 (SLC25A3), H3 histone family 3A (H₃FA), normal mucosa of esophagus specific 1 (NMES1), heat shock 70 kDa protein 8 (HSPA8), hypothetical protein MGC14697 (MGC14697), polymeric immunoglobulin receptor (PIGR), and FK 506 binding protein 8 (FKBP8), hypothetical protein BC012775 (LOC91300), cold shock domain protein A (CSDA), F-box only protein 7 (FBX07), CGI-45 protein (CGI-45), makorin ring finger protein 1 (MKRN1), small EDRK-rich factor 2 (SERF2), pinin (PNN), SET domain bifurcated 1 anti-oxidant protein 2 (AOP2, SETDB1), nuclease sensitive element binding protein (NSEP1), glutathione peroxidase 1 (GPX1), MAX interacting protein 1 (MXI1), and ubiquitin B (UBB).

17. An improved method of analyzing gene expression in a cell or tissue sample, the improvement comprising removing one or more transcripts, prior to or during a reverse transcription reaction, that skew the relative gene expression profile of the cell or tissue sample.

18. The method of claim 17, wherein said one or more transcripts are removed by contacting said one or more transcripts with one or more transcript sequence-specific interfering molecules, or by hybridizing with one or more transcript sequence-specific nucleic acid molecules attached to magnetic beads, wherein said one or more transcript sequence-specific interfering molecules are capable of blocking reverse transcription of said one or more transcripts that skew the relative gene expression profile of the cell or tissue sample.

19. The method of claim 17, wherein the improvement further comprises obtaining a gene expression profile wherein the number of detectable genes obtained is higher than the number of detectable genes obtained when reverse transcription of the unwanted transcript or transcripts is not inhibited.

20. A method for inhibiting amplification of one or more globin mRNA molecules in a sample containing RNA during a nucleic acid amplification process, comprising:
(a) adding one or more globin nucleic acid sequence-specific interfering molecules to the sample; and
(b) amplifying said RNA in the sample in the presence of said one or more globin nucleic acid sequence-specific interfering molecules.

21. The method of claim 20, wherein said globin mRNA molecules are selected from the group consisting of alpha, beta, gamma, delta, theta and zeta globin and variants thereof.

22. The method of claim 20, wherein said sample is a RNA preparation obtained from whole blood, or from a tissue having a high erythrocyte content, wherein said tissue is optionally selected from the group consisting of spleen, bone marrow, placenta, vascularized tumor, angioid tumor, adipose, lung, muscle, pancreas, liver, heart and hemorrhagic tissues.

23. The method of claim 20, wherein said one or more globin nucleic acid sequence-specific interfering molecules have complementarity to globin mRNA, globin cDNA or globin cRNA.

24. The method of claim 23, wherein said one or more globin nucleic acid sequence-specific interfering molecules inhibit amplification of globin mRNA by interfering with a reverse transcriptase or RNA polymerase reaction.

25. The method of claim 24, wherein said one or more globin nucleic acid sequence-specific interfering molecules block reverse transcription of a globin mRNA into a globin cDNA and/or block polymerization of a globin cRNA or cDNA second strand from a globin cDNA.

26. The method of claim 20, wherein said one or more globin nucleic acid sequence-specific interfering molecules are selected from the group consisting of modified and unmodified antisense molecules and triplex forming oligomers.

27. The method of claim 26, wherein said modified anti sense molecules contain one or more modifications selected from the group consisting of nitrogenous base (heterocycle) modifications, sugar modifications, backbone modifications, terminal modifications and functional modifications that result in cleavage of said globin mRNA.

28. The method of claim 27, wherein said one or more sugar modifications are selected from the group consisting of 2′O-alkyl and -halide modifications, carbocyclic sugar mimics and bicyclic sugars, wherein said one or more backbone modifications are selected from the group consisting of phosphorothioate, diphosphorothioate, phosphoroamidate and methylphosphonate modifications, PNAs, 2′-5′ linked oligomers, alpha-linked oligomers, borano-phosphate modified oligomers, chimeric oligomers, anionic, cationic and neutral backbone structures, and wherein said functional modifications are selected from the group consisting of RNAse attachments, ribozyme attachments, chemical group attachments that may be activated to cleave globin mRNA and attachments that lock down the molecule thereby preventing reverse transcriptase or polymerase from melting off the molecule off the globin RNA, wherein said chemical group attachments are optionally selected from the group consisting of aldolating agents, alkylating agents, psoralen and EDTA.

29. The method of claim 20, wherein said one or more globin nucleic acid sequence-specific interfering molecules inhibit amplification of globin mRNA by supporting degradation or cleavage of globin mRNA or cRNA, wherein said degradation or cleavage is optionally caused by RNAse activity or by ribozyme activity.

30. The method of claim 24, wherein said one or more globin nucleic acid sequence-specific interfering molecules are further used to support degradation or cleavage of globin mRNA or cRNA.

31. The method of claim 21, wherein said globin mRNA molecules are from a spec ies selected from the group consisting of human, rat, murine, rabbit, guinea pig, dog, cat, primate, equine, bovine, porcine, ovine and chicken.

32. The method of claim 31, wherein said globin mRNA molecules are human alpha globin mRNA molecules selected from the group consisting of SEQ ID No. 4, SEQ ID No. 5, SEQ ID No. 6 and SEQ ID No. 34, and/or human beta globin mRNA molecules selected from the group consisting of SEQ ID No. 1, SEQ ID No. 2, SEQ ID No. 3, SEQ ID No. 19 and SEQ ID No. 44, and/or human gamma globin mRNA molecules selected from the group consisting of SEQ ID No. 7, SEQ ID No. 8 and SEQ ID No. 9.

33. The method of claim 31, wherein said globin mRNA molecules are rat alpha globin mRNA molecules selected from the group consisting of SEQ ID No. 16, SEQ ID No. 17, SEQ ID No. 18, SEQ ID No. 45, SEQ ID No. 46, SEQ ID No. 47, SEQ ID No. 48 and SEQ ID No. 49, and/or rat beta globin mRNA molecules selected from the group consisting of SEQ ID No. 10, SEQ ID No. 11, SEQ ID No. 12, SEQ ID No. 13, SEQ ID No. 14 and SEQ ID No. 15.

34. The method of claim 20, wherein the RNA is used to obtain a gene expression profile, wherein the gene expression profile is improved as compared to a gene expression profile obtained in the absence of said one or more globin nucleic acid sequence-specific interfering molecules.

35. A kit for inhibiting amplification of one or more globin mRNA molecules in a sample containing RNA during a nucleic acid amplification process, comprising one or more globin nucleic acid sequence-specific interfering molecules.

36. The kit of claim 35, wherein said globin mRNA molecules are selected from the group consisting of alpha, beta, gamma, delta, theta and zeta globin.

37. The kit of claim 35, wherein said one or more globin nucleic acid sequence-specific interfering molecules have complementarity to globin mRNA, globin cDNA or globin cRNA.

38. The kit of claim 35, wherein said one or more globin nucleic acid sequence-specific interfering molecules inhibit amplification of globin mRNA by interfering with a reverse transcriptase or RNA polymerase reaction.

39. The kit of claim 38, wherein said one or more globin nucleic acid sequence-specific interfering molecules block reverse transcription of a globin mRNA into a globin cDNA and/or block polymerization of a globin cRNA or cDNA second strand from a globin cDNA.

40. The kit of claim 35, wherein said one or more globin nucleic acid sequence-specific interfering molecules are selected from the group consisting of modified and unmodified antisense molecules and triplex forming oligomers.

41. The kit of claim 40, wherein said modified antisense molecules contain one or more modifications selected from the group consisting of nitrogenous base (heterocycle) modifications, sugar modifications, backbone modifications, terminal modifications and functional modifications that result in cleavage of said globin mRNA.

42. The kit of claim 41, wherein said one or more sugar modifications are selected from the group consisting of 2′O-alkyl and halide modifications, carbocyclic sugar mimics and bicyclic sugars, wherein said one or more backbone modifications are selected from the group consisting of phosphorothioate, diphosphorothioate, phosphoroamidate and methylphosphonate modifications, PNAs, 2′-5′ linked oligomers, alpha-linked oligomers, borano-phosphate modified oligomers, chimeric oligomers, and anionic, cationic and neutral backbone structures, and wherein said functional modifications are selected from the group consisting of RNase attachments, ribozyme attachments and chemical group attachments that may be activated to cleave globin mRNA, wherein said chemical group attachments are optionally selected from the group consisting of aldolating agents, alkylating agents, psoralen and EDTA.

43. The kit of claim 37, wherein said one or more globin nucleic acid sequence-specific interfering molecules inhibit amplification of globin mRNA by supporting degradation or cleavage of globin mRNA or cRNA, wherein said degradation or cleavage is optionally caused by RNAse activity or by ribozyme activity.

44. The kit of claim 38, wherein said one or more globin nucleic acid sequence-specific interfering molecules are further used to support degradation or cleavage of globin mRNA or cRNA.

45. The kit of claim 35, wherein said globin mRNA molecules are from a species selected from the group consisting of human, rat, murine, rabbit, guinea pig, dog, cat, primate, equine, bovine, porcine, ovine and chicken.

46. The kit of claim 45, wherein said globin mRNA molecules are human alpha globin mRNA molecules selected from the group consisting of SEQ ID No. 4, SEQ ID No. 5, SEQ ID No. 6 and SEQ ID No. 34, and/or human beta globin mRNA molecules selected from the group consisting of SEQ ID No. 1, SEQ ID No. 2, SEQ ID No. 3, SEQ ID No. 19 and SEQ ID No. 44, and/or human gamma globin mRNA molecules selected from the group consisting of SEQ ID No. 7, SEQ ID No. 8 and SEQ ID No. 9.

47. The kit of claim 45, wherein said globin mRNA molecules are rat alpha globin mRNA molecules selected from the group consisting of SEQ ID No. 16, SEQ ID No. 17, SEQ ID No. 18, SEQ ID No. 45, SEQ ID No. 46, SEQ ID No. 47, SEQ ID No. 48 and SEQ ID No. 49, and/or rat beta globin mRNA molecules selected from the group consisting of SEQ ID No. 10, SEQ ID No. 11, SEQ ID No. 12, SEQ ID No. 13, SEQ ID No. 14 and SEQ ID No. 15.

48. A composition useful for obtaining an improved gene expression profile of a cell or tissue sample, comprising one or more interfering molecules specific for the sequences of one or more transcripts that skew the relative gene expression profile of the cell or tissue sample.

49. The composition of claim 48, wherein said one or more interfering molecules are at least 90% identical to said one or more transcripts, or at least 90% identical to nucleic acids that are complementary to said one or more transcripts.

50. The composition of claim 48, wherein said sample is whole blood, and wherein said one or more transcripts are red blood cell transcripts.

51. The composition of claim 50, wherein said one or more red blood cell transcripts are globin transcripts, and wherein said one or more globin transcripts are optionally selected from the group consisting of alpha, beta, gamma, delta, theta and zeta globin and variants thereof.

52. The composition of claim 48, wherein said one or more interfering molecules are selected from the group consisting of modified and unmodified antisense molecules and triplex forming oligomers.

53. The composition of claim 52, wherein said modified antisense molecules contain one or more modifications selected from the group consisting of nitrogenous base (heterocycle) modifications, sugar modifications, backbone modifications, terminal modifications and functional modifications that result in cleavage of said globin mRNA.

54. The composition of claim 53, wherein said one or more sugar modifications are selected from the group consisting of 2′O-alkyl and -halide modifications, carbocyclic sugar mimics and bicyclic sugars, wherein said one or more backbone modifications are selected from the group consisting of phosphorothioate, diphosphorothioate, phosphoroamidate and methylphosphonate modifications, PNAs, 2′-5′ linked oligomers, alpha-linked oligomers, borano-phosphate modified oligomers, chimeric oligomers, anionic, cationic and neutral backbone structures, wherein said functional modifications are selected from the group consisting of RNAse attachments, ribozyme attachments, chemical group attachments that may be activated to cleave globin mRNA and attachments that lock down the molecule thereby preventing reverse transcriptase or polymerase from melting off the molecule off the globin RNA, wherein said chemical group attachments are optionally selected from the group consisting of aldolating agents, alkylating agents, psoralen and EDTA.

55. The composition of claim 48, wherein said one or more interfering molecules have complementarity to globin mRNA, globin cDNA or globin cRNA.

56. The composition of claim 55, wherein said one or more interfering molecules inhibit amplification of globin mRNA by supporting degradation or cleavage of globin mRNA or cRNA, wherein said degradation or cleavage is optionally caused by RNAse activity or by ribozyme activity.

57. The composition of claim 48, wherein said globin mRNA molecules are from a species selected from the group consisting of human, rat, murine, rabbit, guinea pig, dog, cat, primate, equine, bovine, porcine, ovine and chicken.

58. The composition of claim 57, wherein said globin mRNA molecules are human alpha globin mRNA molecules selected from the group consisting of SEQ ID No. 4, SEQ ID No. 5, SEQ ID No. 6 and SEQ ID No. 34, and/or human beta globin mRNA molecules selected from the group consisting of SEQ ID No. 1, SEQ ID No. 2, SEQ ID No. 3, SEQ ID No. 19 and SEQ ID No. 44, and/or human gamma globin mRNA molecules selected from the group consisting of SEQ ID No. 7, SEQ ID No. 8 and SEQ ID No. 9.

59. The composition of claim 57, wherein said globin mRNA molecules are rat alpha globin mRNA molecules selected from the group consisting of SEQ ID No. 16, SEQ ID No. 17, SEQ ID No. 18, SEQ ID No. 45, SEQ ID No. 46, SEQ ID No. 47, SEQ ID No. 48 and SEQ ID No. 49, and/or rat beta globin mRNA molecules selected from the group consisting of SEQ ID No. 10, SEQ ID No. 11, SEQ ID No. 12, SEQ ID No. 13, SEQ ID No. 14 and SEQ ID No. 15.

60. The method of claim 1 wherein the sequence-specific interfering molecule is a gene specific primer that substantially blocks reverse transcription of mRNA.

61. The method of claim 60, wherein the primer has a high G/C content.

62. The method of claim 61, wherein the primer is extended in the 3′ direction by reverse transcriptase and blocks synthesis of a cDNA strand from a transcription initiation primer.

63. The kit of claim 35, wherein the sequence-specific interfering molecule is a gene specific primer that substantially blocks reverse transcription of mRNA.

64. The kit of claim 63, wherein the primer has a high G/C content.

65. The kit of claim 64, wherein the primer is extended in the 3′ direction by reverse transcriptase and blocks synthesis of a cDNA strand from a transcription initiation primer.

66. The composition of claim 48, wherein the interfering molecule is a gene-specific primer that substantially blocks reverse transcription of said one or more transcripts.

67. The composition of claim 66, wherein the primer has a high G/C content.

68. The composition of claim 67, wherein the primer is extended in the 3′ direction by reverse transcriptase and blocks synthesis of a cDNA strand from a transcription initiation primer.

69. The method of claim 1 wherein said amplification comprises reverse transcription of said transcript species.


SEQUENCE LISTING (49 sequences)

SEQ ID NO: 1 - RNA, 20 nt, Homo sapiens
gcagaauccagaugcucaag

SEQ ID NO: 2 - RNA, 23 nt, Homo sapiens
  misc_feature (1)..(23): May be 2′-O-methyl bases
ggacagcaagaaagcgagcuuug

SEQ ID NO: 3 - RNA, 21 nt, Homo sapiens
cauugagccacaccagccacc

SEQ ID NO: 4 - RNA, 20 nt, Homo sapiens
uuugccgcccacucagacuu

SEQ ID NO: 5 - RNA, 23 nt, Homo sapiens
  misc_feature (1)..(23): May be 2′-O-methyl bases
ccaccgaggcuccagcuuaacgg

SEQ ID NO: 6 - RNA, 23 nt, Homo sapiens
guccacccgaagcuugugcgcgu

SEQ ID NO: 7 - RNA, 23 nt, Homo sapiens
  misc_feature (1)..(23): May be 2′-O-methyl bases
ugugaucucucagcagaauagau

SEQ ID NO: 8 - RNA, 23 nt, Homo sapiens
gccuauccuugaaagcucugaau

SEQ ID NO: 9 - RNA, 23 nt, Homo sapiens
ccacugcagucaccaucuucugc

SEQ ID NO: 10 - RNA, 20 nt, Rattus sp.
  misc_feature (1)..(20): May be 2′-O-methyl bases
gcagugaaaguaaaugccuu

SEQ ID NO: 11 - RNA, 23 nt, Rattus sp.
  misc_feature (1)..(23): May be 2′-O-methyl bases
gacaacaacugacagaugcucuc

SEQ ID NO: 12 - RNA, 25 nt, Rattus sp.
ccaccuucuggaaggcagccugugc

SEQ ID NO: 13 - RNA, 22 nt, Rattus sp.
  misc_feature (1)..(22): May be 2′-O-methyl bases
gcucucuugggaacaauugacc

SEQ ID NO: 14 - RNA, 22 nt, Rattus sp.
  misc_feature (1)..(22): May be 2′-O-methyl bases
ggcacuggccacuccagccacc

SEQ ID NO: 15 - RNA, 21 nt, Rattus sp.
ccaggagccugaaguucucag

SEQ ID NO: 16 - RNA, 19 nt, Rattus sp.
uugcuuccuacucaggcuu

SEQ ID NO: 17 - RNA, 22 nt, Rattus sp.
agagguauaggugcaagggagg

SEQ ID NO: 18 - RNA, 22 nt, Rattus sp.
ggucagcacagugcucacagag

SEQ ID NO: 19 - RNA, 23 nt, Homo sapiens
gcauuagccacaccagccaccac

SEQ ID NO: 20 - RNA, 25 nt, Homo sapiens
ugaaguugagcugaacauucuuuau

SEQ ID NO: 21 - RNA, 24 nt, Homo sapiens
gcagaagccauacccuugaaguag

SEQ ID NO: 22 - RNA, 23 nt, Homo sapiens
guguucccaaguucagaaaauag

SEQ ID NO: 23 - RNA, 26 nt, Homo sapiens
guuaucaggaaacaguccaggaucuc

SEQ ID NO: 24 - RNA, 24 nt, Canis sp.
gcgaagaacuuguccagguaggcg

SEQ ID NO: 25 - RNA, 24 nt, Canis sp.
cuuccaguggucaccaggaaacag

SEQ ID NO: 26 - RNA, 26 nt, Unknown Sequence
  FK 506 Binding Protein 8 interfering molecule
gaagggcugcccccaggccuguugag

SEQ ID NO: 27 - RNA, 29 nt, Unknown Sequence
  FK 506 Binding Protein 8 interfering molecule
gaggccagcccuggcggagaccuagccca

SEQ ID NO: 28 - RNA, 23 nt, Unknown Sequence
  FK 506 Binding Protein 8 interfering molecule
ccucugggcuuuccuccuagagg

SEQ ID NO: 29 - RNA, 24 nt, Unknown Sequence
  FK 506 Binding Protein 8 interfering molecule
ccugcuggcugggcugcacgaccc

SEQ ID NO: 30 - RNA, 23 nt, Unknown Sequence
  Selenium Binding Protein 1 interfering molecule
cagcacagugagcaacaagcaac

SEQ ID NO: 31 - RNA, 25 nt, Unknown Sequence
  Selenium Binding Protein 1 interfering molecule
cuuggugccuccaagagcugccaag

SEQ ID NO: 32 - RNA, 24 nt, Unknown Sequence
  Selenium Binding Protein 1 interfering molecule
caagagagagcagaaugaagccag

SEQ ID NO: 33 - RNA, 23 nt, Unknown Sequence
  Selenium Binding Protein 1 interfering molecule
gugaugaggguggaguucaaauc

SEQ ID NO: 34 - RNA, 23 nt, Homo sapiens
  misc_feature (1)..(23): May be 2′-O-methyl bases
uuugccgcccacucagacuuuau

SEQ ID NO: 35 - RNA, 23 nt, Macaca fascicularis
  modified_base (1)..(23): 2′-O-methyl bases
ggcagaauccagauccucaaggg

SEQ ID NO: 36 - RNA, 22 nt, Macaca fascicularis
  modified_base (1)..(22): 2′-O-methyl bases
cauaauaucccccaguucagug

SEQ ID NO: 37 - RNA, 23 nt, Macaca fascicularis
  modified_base (1)..(23): 2′-O-methyl bases
ggacagcaagaaagugagcuuug

SEQ ID NO: 38 - RNA, 26 nt, Canis sp.
  modified_base (1)..(26): 2′-O-methyl bases
gcaggcagcccacucagacuuuauuc

SEQ ID NO: 39 - RNA, 26 nt, Canis sp.
  modified_base (1)..(26): 2′-O-methyl bases
ucaaacaucaggaagugcagggcacc

SEQ ID NO: 40 - RNA, 24 nt, Canis sp.
  modified_base (1)..(24): 2′-O-methyl bases
gcgcaggaagcggcccagggcagg

SEQ ID NO: 41 - RNA, 24 nt, Canis sp.
  modified_base (1)..(24): 2′-O-methyl bases
gaagccauacccuugaugguagac

SEQ ID NO: 42 - RNA, 24 nt, Canis sp.
  modified_base (1)..(24): 2′-O-methyl bases
cuuccaguggucaccaggaaacag

SEQ ID NO: 43 - RNA, 23 nt, Canis sp.
  modified_base (1)..(23): 2′-O-methyl bases
gccacaccagccaccaccuucug

SEQ ID NO: 44 - RNA, 23 nt, Homo sapiens
  modified_base (1)..(23): 2′-O-methyl bases
ggcagaauccagaugcucaaggc

SEQ ID NO: 45 - RNA, 23 nt, Rattus sp.
  misc_feature (1)..(23): May be 2′-O-methyl bases
ggacggaagaagggccuggucag

SEQ ID NO: 46 - RNA, 22 nt, Rattus sp.
  misc_feature (1)..(22): May be 2′-O-methyl bases
gcaagcccgacaggagguggcu

SEQ ID NO: 47 - RNA, 24 nt, Rattus sp.
cagagucuucuuuccuaguucugc

SEQ ID NO: 48 - RNA, 22 nt, Rattus sp.
cuuugcacaugcauauaaauag

SEQ ID NO: 49 - RNA, 20 nt, Rattus sp.
uuauucaaauacugguucag